(19) United States
(12) Patent Application Publication
(10) Pub. No.: US 2008/0305501 A1
(43) Pub. Date: Dec. 11, 2008

(54) Compositions and Methods for Modulating Rank Activities

(76) Inventors: Xu Feng, Birmingham, AL (US); Duorong Xu, Guangzhou (CN); Shunqing Wang, Homewood, AL (US)

Correspondence Address: SUTHERLAND ASBILL & BRENNAN LLP, 999 PEACHTREE STREET, N.E., ATLANTA, GA, 30309, US

(21) Appl. No.: 11/997,862

(22) PCT Filed: Aug. 2, 2006
(86) PCT No.: PCT/US06/30021

§ 371 Date: May 15, 2008

Related U.S. Application Data
(60) Provisional application No. 60/704,828, filed on Aug. 2, 2005.

(51) Int. Cl. G01N 33/567; C07K 14/705; C07H 21/04

(52) U.S. Cl. 435/7.21; 530/350; 536/23.5

(57) ABSTRACT

The present invention provides methods for identifying agents capable of modulating RANK-mediated invention also provides pharmaceutical compositions and methods of using the same for treating osteoporosis or other diseases. The present invention is based on the functional and structural analysis of a novel RANK signaling motif that was found to play a distinct role in activating RANK-mediated intracellular signaling. This motif can be used to screen for RANK modulators. This motif for treating a variety of diseases that are caused by or associated with abnormal RANK expression or activities.

### FIELD OF THE INVENTION

The present invention relates to the structure and function of a novel RANK signaling motif and methods of using this motif to identify agents that modulate RANK activities. The present invention also relates to compositions and methods for treating osteoporosis or other RANK-associated diseases.

### BACKGROUND OF THE INVENTION

Osteoclasts, the principal bone-resorbing cells, play a pivotal role in skeleton development and maintenance. Osteoclasts are derived from mononuclear precursors of monocyte/macrophage lineage upon stimulation of two key factors: monocyte/macrophage colony stimulating factor (M-CSF) and receptor activator of nuclear factor kappa B (RANKL, also known as OPGL/ODF/TRANCE). RANKL, a member of the tumor necrosis factor (TNF) superfamily, regulates both osteoclast formation and function by binding to its receptor RANK expressed on osteoclast precursors and mature osteoclasts. The essential role of RANKL and RANK in the osteoclastogenic process has been demonstrated by the findings that mice lacking the gene for either protein develop osteopetrosis due to failure to form osteoclasts.

RANK (receptor activator of NF-κB) was identified as a member of the TNF receptor family. Members of the TNF receptor family are characterized by lack of intrinsic enzymatic activity and thus they usually transduce intracellular signals by recruiting various adaptor proteins such as TNF receptor associated factors (TRAFs) through the specific motifs in their cytoplasmic domains. Since the unraveling of the RANKL/RANK system, efforts have been undertaken to elucidate RANK-initiated intracellular signaling. Many of the previous works have been focused on characterizing the receptor-proximal signaling events, which represent the initial components of intracellular signaling pathways initiated by membrane-bound receptors. Although these studies have mapped RANK cytoplasmic regions capable of interacting with TRAF proteins by various in vitro binding assays, the physiological relevance of these data to osteoclast biology remained largely unexplored. A recent functional study confirmed that RANK contains three TRAF-binding sites that play redundant role in osteoclast formation and function (Liu et al, (2004) J. Biol. Chem. 279, 54759-54769). Using these TRAF proteins, RANK has been shown to activate six major signaling pathways: NF-κB, JNK, ERK, p38, NFATc1, AKT and c-fos (Boyle et al., (2003) Nature 423, 337-342; Takayanagi et al., (2002) Nature 416, 744-749).

However, several lines of evidence suggest that RANK may also activate novel pathways to regulate osteoclast formation. For example, although it has been established that, like RANK, IL-1R utilizes TRAF6 to activate intracellular signaling pathways (Inoue et al., (2000) Exp. Cell Res. 254, 14-24), administration of IL-1 to RANK^{-/-} mice fails to promote osteoclast formation (Li et al., (2000) Proc. Natl. Acad. Sci. U.S.A. 97, 1566-1571). The complete absence of osteoclasts in the RANK^{-/-} mice administrated with IL-1 suggests that RANK utilizes other novel signaling pathways in mediating osteoclast formation. Similarly, two other reports demonstrated that IL-1 fails to stimulate osteoclast formation in vitro even in the presence of M-CSF (Azuma et al., (2000) J. Biol. Chem. 275, 4858-4864; Kobayashi et al., (2000) J. Exp. Med. 191, 275-28520; 21).

In addition, the discovery of the RANKL/RANK/OPG regulatory axis has raised high expectation to develop osteoprotegerin (OPG) and soluble RANK-Fc as therapeutic drugs to treat bone diseases. However, both OPG and RANK-Fc have a possible drawback, primarily due to the fact that their action lacks specificity. RANKL not only plays a pivotal role in osteoclast formation and differentiation, but also functions as a critical mediator in other biological processes such as the immune system and mammary gland development. As a result, use of either OPG or RANK-Fc to treat bone diseases may cause potential adverse effect on patients' immune system. There is therefore a need to develop new therapeutics to affect osteoclast differentiation and function.

### SUMMARY OF THE INVENTION

The present invention characterizes the RANK-initiated signaling in physiological cellular background and identifies a specific RANK motif that regulates osteoclast formation and function. The identification of this functional RANK motif has laid foundations for further delineating the downstream signaling pathways implicated in osteoclast formation and function.

In one aspect, the invention encompasses a variant RANK polypeptide comprising at least one mutation at an amino acid residue corresponding to an amino acid residue selected from the group consisting of I535, V536, V537, Y538, and any combination of the foregoing, of SEQ ID NO:2. In one embodiment, the variant RANK polypeptide has decreased activity of at least one RANK-mediated signaling pathway. In another embodiment, when expressed in an osteoclast precursor cell, the variant RANK polypeptide decreases osteoclast formation. The invention also encompasses a nucleic acid encoding a RANK polypeptide comprising at least one mutation at an amino acid residue corresponding to an amino acid residue selected from the group consisting of I535, V536, V537, Y538, and any combination of the foregoing, of SEQ ID NO:2. In one embodiment, the invention is directed to a chimeric polypeptide comprising an non-RANK extracellular domain and at least 20 contiguous amino acids of a RANK intracellular domain, comprising residues corresponding to amino acids 535-538 of SEQ ID NO:2. In one embodiment, the non-RANK extracellular domain is a TNFR1 extracellular domain. In a further embodiment, the RANK intracellular domain comprises at least one mutation at an amino acid residue corresponding to an amino acid residue selected from the group consisting of I535, V536, V537, Y538, and any combination of the foregoing, of SEQ ID NO:2.

In another aspect, the present invention provides methods for identifying compounds capable of modulating osteoclast differentiation. One such method comprises (a) providing an osteoclast precursor cell comprising a receptor comprising a RANK polypeptide, (b) contacting the osteoclast precursor cell with a test compound and a ligand for the receptor, wherein the test compound interacts with one or more amino acids corresponding to an amino acid residue of 535-538 of SEQ ID NO:2, and (c) determining whether osteoclast formation has been modulated, said modulation being an indication that the compound modulates osteoclast cell differentiation.

In yet another aspect, the present invention provides methods for identifying compounds capable of modulating RANK activity. One method comprises (a) inducing oligomerization of a receptor comprising a RANK polypeptide in the presence or absence of a test compound, wherein the test compound interacts with one or more amino acids corresponding to an amino acid residue of 535-538 of SEQ ID NO:2, and (b) detecting modulation of at least one RANK-mediated signaling pathway in said cell after said oligomerization, said activation level being an indication that the compound modulates activity of RANK. Another method comprises (a) providing a cell comprising a receptor comprising a RANK polypeptide, (b) contacting the cell with a test compound and a ligand for the receptor, wherein the test compound interacts with one or more amino acids corresponding to an amino acid residue of 535-538 of SEQ ID NO:2, and (c) determining whether a RANK-mediated signaling pathway has been modulated, said modulation being an indication that the compound modulates RANK activity. A further method comprises (a) inducing oligomerization of a chimeric transmembrane protein in an osteoclast precursor cell in the presence or absence of a test compound, said chimeric protein comprising a non-RANK extracellular domain and a RANK intracellular domain, wherein the test compound interacts with one or more amino acids corresponding to an amino acid residue of 535-538 of SEQ ID NO:2; and (b) detecting activation level of at least one RANK-mediated signaling pathway in said cell after said oligomerization, wherein a reduction in the activity level in the presence of said molecule compared to that in the absence of said molecule is indicative of the ability of said molecule to inhibit RANK activity.

In another aspect, the invention encompasses a process for making a compound that decreases osteoclast cell differentiation, comprising carrying out any of the methods described herein to identify a compound that decreases osteoclast cell differentiation, and manufacturing the compound.

The present invention also features methods of modulating RANK activity in a cell of interest. The methods include contacting one or more compounds with the cell to modulate at least one RANK-mediated signaling pathway dependent on the novel motif identified herein.

It is contemplated that such compounds can be administered to individuals in order to treat bone loss. As such, the invention provides for a method of improving bone mass in an individual in need thereof, comprising administering to the individual a therapeutically effective amount of a compound that decreases differentiation of a osteoclast precursor cell to an osteoclast, wherein the compound interacts with one or more amino acids corresponding to residues 535-538 of SEQ ID NO:2. In another embodiment, the invention provides a method of improving bone mass in an individual in need thereof, comprising administering to the individual a therapeutically effective amount of a compound that inhibits the activity of RANK in an osteoclast cell, wherein the compound interacts with one or more amino acids corresponding to residues 535-538 of SEQ ID NO:2. An individual in need of improving bone mass typically has a bone-related disorder, such as, but not limited to osteoporosis.

In certain embodiments, the RANK polypeptide is selected from the group consisting of a mammalian polypeptide. It is contemplated that the RANK polypeptide is a mouse polypeptide. It is also contemplated that that RANK polypeptide is a human polypeptide.

The invention encompasses the use of a receptor that is a chimeric polypeptide. In one embodiment, the chimeric polypeptide comprises a RANK intracellular domain. In a further embodiment, the chimeric polypeptide comprises at least contiguous 20 amino acids of a RANK polypeptide, wherein the RANK polypeptide comprises residues corresponding to amino acids 535-538 of SEQ ID NO:2. In another embodiment, the chimeric polypeptide comprises a non-RANK polypeptide comprising an extracellular domain. In one embodiment, the non-RANK polypeptide is a TNF receptor, and the ligand is TNFα.

Oligomerization of the receptor can occur within a cell. In one embodiment, the cell is an osteoclast precursor cell. In a further embodiment, the activation level of the RANK-mediated signaling pathway is determined by detecting osteoclast formation. In one embodiment of the invention, the RANK-mediated signaling pathway is down-regulated, and osteoclast formation is decreased.

Other features, and advantages of the invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1A shows a schematic diagram of 20 internal deletion mutants: D1-D20. Ext: external domain; TM: transmembrane domain. FIG. 1B shows osteoclast formation assays with the mutants. TNFR1^{-/-} TNFR 2^{-/-} BMMs were either uninfected or infected with virus encoding wild-type chimera (WT), or mutants (D1-D20), and were selected with puromycin for 2 days. Uninfected BMMs or infected BMMs were then treated with M-CSF (22 ng/ml) and TNFα (10 ng/ml). Osteoclasts began to form at day 3 and the cultures were stained for TRAP activity at day 6. FIG. 1C depicts flow cytometric analysis showing the surface expression of the chimeras on the infected BMMs using TNFR1 antibody conjugated with phycoerythrin (Santa Cruz, Calif., sc-12746PE). Uninfected BMMs were used as control.

FIG. 2A shows the sequence and location of a 40-a.a. segment in the mouse RANK cytoplasmic domain (residues 513-552 of SEQ ID NO:2). TM: transmembrane domain. FIG. 2B depicts a sequence comparison of mouse and human RANK cytoplasmic domain, comparing residues 235-612 of SEQ ID NO:2 to residues 234-616 of SEQ ID NO:3, respectively. The three boxed sequences are TRAF-binding sites. The underlined region is the 40-a.a. segment essential for osteoclast formation.

FIG. 3A shows a schematic diagram of 10 internal deletion mutants generated, in which a 4-a.a. segment of SEQ ID NO:2 was deleted in each mutant. The internal deletion mutants were designated SD1-SD10. FIG. 3B depicts the results of osteoclast formation assays performed with SD1-SD10. Infected BMMs were treated with 22 ng/ml of M-CSF plus 10 ng/ml of TNFα. FIG. 3C depicts the results of osteoclast formation assays with SD4, SD5, SD6 and SD7. Infected BMMs were treated with 22 ng/ml of M-CSF plus 30 ng/ml of TNFα. FIG. 3D depicts flow cytometric analysis showing the surface expression of the mutants on the infected BMMs.

FIG. 4A shows a schematic diagram of 4 point mutation mutants, designated PM1-PM4. The sequence DIIVVYVS (residues 533-540 of SEQ ID NO:2) was mutated to ELIVVYVS (SEQ ID NO:4), DILAVYVS (SEQ ID NO:5), DIIVAFVS (SEQ ID NO:6), and DIIVVYAA (SEQ ID NO:7). FIG. 4B shows the results of osteoclast formation assays with PM1-PM4. Infected BMMs were treated with 22 ng/ml of M-CSF plus 10 ng/ml of TNFα. FIG. 4C depicts flow cytometric analysis showing the surface expression of the mutants on the infected BMMs.

FIGS. 5A-E show Western blots of uninfected BMMs, and BMMs infected with wild-type and PM3, demonstrating activation of NF-κB/IκB, JNK, ERK, p38 and Akt pathways by the wild-type chimera and PM3. Activation of these signaling pathways was assessed by phosphorylation of NF-KB/IκB (FIG. 5A), JNK (FIG. 5B), ERK (FIG. 5C), p38 (FIG. 5D) and Akt (FIG. 5E) using Western analysis with antibodies against phospho-IκB, phospho-JNK, phospho-ERK, phospho-p38 and phosphor-Akt.

FIG. 6A is a schematic depicting the strategy used to examine the role of the novel motif in osteoclast function and survival in TNFR1^{-/-}R2^{-/-} infected BMMs. FIG. 6B depicts bone resorption assays showing that osteoclasts expressing either WT chimera or PM3 were very efficient in mediating bone resorption in response to TNFα stimulation. FIG. 6C depicts osteoclast survival assays showing that osteoclasts expressing WT chimera or PM3 have a similar ability to promote osteoclast survival in response to TNFα stimulation.

FIG. 7A is a schematic depicting the strategy used to examine the role of the novel motif in commitment to the osteoclast lineage in TNFR1^{-/-}R2^{-/-} infected BMMs. BMMs were treated with varying amounts of time with M-CSF (44 ng/ml) and RANKL (100 ng/ml) (R), and were then treated with M-CSF (44 ng/ml) and TNF-α (10 ng/ml) (T) for the rest of the osteoclastogenic process. In assay 1, the cells were only treated with M-CSF (44 ng/ml) and TNF-α (10 ng/ml) throughout the 6 days of the osteoclastogenic process. FIG. 7B depicts osteoclast formation assays, showing that treatment of BMMs with RANKL for only 4 hours can partially commit BMMs to osteoclast lineage. Moreover, 16 or 24-hour treatment of BMMs with RANKL can fully commit BMMs to osteoclast lineage.

### DETAILED DESCRIPTION OF THE INVENTION

The present invention may be understood more readily by reference to the following detailed description of the preferred embodiments of the invention and the Examples included herein. However, before the present compositions and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

The present invention relates to the characterization of the RANK-initiated signaling in physiological cellular background and identified a specific RANK motif that regulates osteoclast formation and function. The identification of this functional RANK motif has laid foundations for further delineating the downstream signaling pathways implicated in osteoclast formation and function.

In one aspect, the invention encompasses a variant RANK polypeptide comprising at least one mutation at an amino acid residue corresponding to an amino acid residue selected from the group consisting of I535, V536, V537, Y538, and any combination of the foregoing, of SEQ ID NO:2. In one embodiment, the variant RANK polypeptide has decreased activity of at least one RANK-mediated signaling pathway. In another embodiment, when expressed in an osteoclast precursor cell, the variant RANK polypeptide decreases osteoclast formation. The invention also encompasses a nucleic acid encoding a RANK polypeptide comprising at least one mutation at an amino acid residue corresponding to an amino acid residue selected from the group consisting of I535, V536, V537, Y538, and any combination of the foregoing, of SEQ ID NO:2. In one embodiment, the invention is directed to a chimeric polypeptide comprising an non-RANK extracellular domain and at least 20 contiguous amino acids of a RANK intracellular domain, comprising residues corresponding to amino acids 535-538 of SEQ ID NO:2. In one embodiment, the non-RANK extracellular domain is a TNFR1 extracellular domain. In a further embodiment, the RANK intracellular domain comprises at least one mutation at an amino acid residue corresponding to an amino acid residue selected from the group consisting of I535, V536, V537, Y538, and any combination of the foregoing, of SEQ ID NO:2.

In another aspect, the present invention provides methods for identifying compounds capable of modulating osteoclast differentiation. One such method comprises (a) providing an osteoclast precursor cell comprising a receptor comprising a RANK polypeptide, (b) contacting the osteoclast precursor cell with a test compound and a ligand for the receptor, wherein the test compound interacts with one or more amino acids corresponding to an amino acid residue of 535-538 of SEQ ID NO:2, and (c) determining whether osteoclast formation has been modulated, said modulation being an indication that the compound modulates osteoclast cell differentiation.

In yet another aspect, the present invention provides methods for identifying compounds capable of modulating RANK activity, the compounds identified therein, and the use of such compounds to treat bone loss. One method comprises (a) inducing oligomerization of a receptor comprising a RANK polypeptide in the presence or absence of a test compound, wherein the test compound interacts with one or more amino acids corresponding to an amino acid residue of 535-538 of SEQ ID NO:2, and (b) detecting modulation of at least one RANK-mediated signaling pathway in said cell after said oligomerization, said activation level being an indication that the compound modulates activity of RANK. Another method comprises (a) providing a cell comprising a receptor comprising a RANK polypeptide, (b) contacting the cell with a test compound and a ligand for the receptor, wherein the test compound interacts with one or more amino acids corresponding to an amino acid residue of 535-538 of SEQ ID NO:2, and (c) determining whether a RANK-mediated signaling pathway has been modulated, said modulation being an indication that the compound modulates RANK activity. A further method comprises (a) inducing oligomerization of a chimeric transmembrane protein in an osteoclast precursor cell in the presence or absence of a test compound, said chimeric protein comprising a non-RANK extracellular domain and a RANK intracellular domain, wherein the test compound interacts with one or more amino acids corresponding to an amino acid residue of 535-538 of SEQ ID NO:2; and (b) detecting activation level of at least one RANK-mediated signaling pathway in said cell after said oligomerization, wherein a reduction in the activity level in the presence of said molecule compared to that in the absence of said molecule is indicative of the ability of said molecule to inhibit RANK activity.

In another aspect, the invention encompasses a process for making a compound that decreases osteoclast cell differentiation, comprising carrying out any of the methods described herein to identify a compound that decreases osteoclast cell differentiation, and manufacturing the compound.

The present invention also features methods of modulating RANK activity in a cell of interest. The methods include contacting one or more compounds with the cell to modulate at least one RANK-mediated signaling pathway dependent on the novel motif identified herein.

It is contemplated that such compounds can be administered to individuals in order to treat bone loss. As such, the invention provides for a method of improving bone mass in an individual in need thereof, comprising administering to the individual a therapeutically effective amount of a compound that decreases differentiation of a osteoclast precursor cell to an osteoclast, wherein the compound interacts with one or more amino acids corresponding to residues 535-538 of SEQ ID NO:2. In another embodiment, the invention provides a method of improving bone mass in an individual in need thereof, comprising administering to the individual a therapeutically effective amount of a compound that inhibits the activity of RANK in an osteoclast cell, wherein the compound interacts with one or more amino acids corresponding to residues 535-538 of SEQ ID NO:2. An individual in need of improving bone mass typically has a bone-related disorder.

In certain embodiments, the RANK polypeptide is selected from the group consisting of a mammalian polypeptide. It is contemplated that the RANK polypeptide is a mouse polypeptide. It is also contemplated that that RANK polypeptide is a human polypeptide.

The invention encompasses the use of a receptor that is a chimeric polypeptide. In one embodiment, the chimeric polypeptide comprises a RANK intracellular domain. In a further embodiment, the chimeric polypeptide comprises at least contiguous 20 amino acids of a RANK polypeptide, wherein the RANK polypeptide comprises residues corresponding to amino acids 535-538 of SEQ ID NO:2. In another embodiment, the chimeric polypeptide comprises a non-RANK polypeptide comprising an extracellular domain. In one embodiment, the non-RANK polypeptide is a TNF receptor, and the ligand is TNFα.

Oligomerization of the receptor can occur within a cell. In one embodiment, the cell is an osteoclast precursor cell. In a further embodiment, the activation level of the RANK-mediated signaling pathway is determined by detecting osteoclast formation. In one embodiment of the invention, the RANK-mediated signaling pathway is down-regulated, and osteoclast formation is decreased.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., (1989) Molecular Cloning, Second Edition, Cold Spring Harbor Laboratory, Plainview, N.Y.; Maniatis et al., (1982) Molecular Cloning, Cold Spring Harbor Laboratory, Plainview, N.Y.; Wu (Ed.) (1993) Meth. Enzymol. 218, Part I; Wu (Ed.) (1979) Meth. Enzymol. 68; Wu et al., (Eds.) (1983) Meth. Enzymol. 100 and 101; Grossman and Moldave (Eds.) (1980) Meth. Enzymol. 65; Miller (ed.) (1972) Experiments in Molecular Genetics, Cold Spring Harbor Laboratory, Cold Spring Harbor, N.Y.; Old and Primrose, (1981) Principles of Gene Manipulation, University of California Press, Berkeley; Schleif and Wensink, (1982) Practical Methods in Molecular Biology; Glover (Ed.) (1985) DNA Cloning Vol. I and II, IRL Press, Oxford, UK; Hames and Higgins (Eds.) (1985) Nucleic Acid Hybridization, IRL Press, Oxford, UK; and Setlow and Hollaender (1979) Genetic Engineering Principles and Methods, Vols. 1-4, Plenum Press, New York. Abbreviations and nomenclature, where employed, are deemed standard in the field and commonly used in professional journals such as those cited herein.

In one aspect, the present invention provides methods for identifying or evaluating agents capable of modulating RANK activities in osteoclasts or other cells. The methods typically include inducing oligomerization of a chimeric or non-chimeric protein in a cell or a cell-free system, and detecting the activities of RANK-mediated signaling pathways in the cell or cell-free system. As used herein, the term “modulator of the RANK-mediated signaling pathway” refers to any compound that increased or decreases the activity of RANK or modulates the activity of at least one molecule downstream of RANK in a cell contacted with the modulator. It is understood that combinations of modulators may be used to elicit the desired effect. It is contemplated that the modulator of RANK-mediated signaling may act directly on RANK or may act on a molecule upstream or downstream of RANK to thereby modulate RANK signaling. In one embodiment, the modulator interacts with the novel motif identified in the current invention to thereby modulate the activity of RANK. As used herein, the term “interact” refers to the direct or indirect interaction of the modulator with one or more amino acids corresponding to amino acid residues 535-538 of SEQ ID NO:2. It is contemplated that such interaction can also involve the interaction with other RANK amino acid residues.

In certain embodiments of the invention, the receptor used in the screen is a transmembrane protein comprising an oligomerizable extracellular domain and an intracellular domain including the novel motif identified herein. Oligomerization of the chimeric protein triggers activation of RANK-mediated signaling pathways. The activation of these pathways can be monitored in the presence or absence of a compound of interest. Compounds capable of inhibiting or otherwise modulating the activities of these pathways can therefore be identified.

In one embodiment, the extracellular domain employed in the present invention is capable of inducing oligomerization (e.g., timerization) of the chimeric protein upon binding to a non-RANKL ligand. Extracellular domains suitable for this purpose include, but are not limited to, the extracellular domains of numerous tumor necrosis factor receptors, such as TNFR1 (tumor necrosis factor receptor superfamily, member 1A), TNFR2 (tumor necrosis factor receptor superfamily, member 1B), or Fas (tumor necrosis factor receptor superfamily, member 6). The extracellular domains of other receptor proteins whose activation is triggered by oligomerization may also be used for the present invention.

It is contemplated that the chimeric proteins employed in the present invention comprise an endogenous RANK cytoplasmic domain. Murine and human RANK proteins have Entrez accession numbers NP_{—}033425 and NP_{—}003830, respectively, and their cytoplasmic domains consist of amino acid 235 to 625 for murine RANK protein, and amino acids 234 to 616 for human murine RANK protein. Cytoplasmic domains of other RANK proteins can also be used in the present invention. RANK genes of other species can be readily identified based on murine or human sequences. Methods suitable for this purpose include, but are not limited to, genetic or cDNA library screens or genome BLAST searches. Genomes of many species are available at Entrez (National Center for Biotechnology Information, Bethesda, Md. 20894). The RANK gene of a species of interest can be identified through BLAST searching the genome of interest by using murine or human sequences as the query sequences. The cytoplasmic domain of a RANK gene thus identified can be determined by using transmembrane prediction programs, such as TMHMM, or any other means known in the art.

In many other embodiments, the chimeric proteins employed in the present invention include one or more fragments of an endogenous RANK cytoplasmic domain. Each fragment includes a motif corresponding to one or more amino acid residues 535-538 of SEQ ID NO:2. A corresponding motif from any species may be used for the present invention. In many examples, the motif from murine or human RANK is employed. The mouse motif for IVVY is found at residues 535-538 of SEQ ID NO:2. The human motif corresponding to mouse residues 535-538 of SEQ ID NO:2 is found at 547-550 of SEQ ID NO:3.

Amino acid residues surrounding residues corresponding to amino acids 535-538 of SEQ ID NO:2 in the endogenous RANK cytoplasmic domain can also be included in the chimeric protein to improve the protein's interaction with downstream signaling molecules. In many cases, the chimeric polypeptide includes an endogenous RANK cytoplasmic sequence consisting of from about 5 to about 10, from about 10 to about 20, or from about 20 to about 30 amino acid residues. A chimeric polypeptide of the present invention can include sequences derived from the same or different species.

The present invention further contemplates the use of non-transmembrane proteins for identifying or evaluating RANK modulators. These non-transmembrane proteins include the novel motif identified herein capable of activating the downstream signaling pathways. In many cases, the non-transmembrane proteins are cytosolic proteins that include a domain that can trigger protein oligomerization upon occurrence of a specified event, such as binding to a ligand or changing in the ionic strength. In many other cases, the novel motif of the invention comprised in a non-transmembrane protein of the present invention can activate RANK signaling pathway(s) without any triggering event.

As described above, the invention encompasses a variant RANK polypeptide comprising at least one mutation at an amino acid residue corresponding to an amino acid residue selected from the group consisting of I535, V536, V537, Y538, and any combination of the foregoing, of SEQ ID NO:2; a nucleic acid encoding a RANK polypeptide comprising at least one mutation at an amino acid residue corresponding to an amino acid residue selected from the group consisting of I535, V536, V537, Y538, and any combination of the foregoing, of SEQ ID NO:2; and a chimeric polypeptide comprising an non-RANK extracellular domain and at least 20 contiguous amino acids of a RANK intracellular domain, comprising residues corresponding to amino acids 535-538 of SEQ ID NO:2. In a further embodiment, the RANK intracellular domain comprises at least one mutation at an amino acid residue corresponding to an amino acid residue selected from the group consisting of I535, V536, V537, Y538, and any combination of the foregoing, of SEQ ID NO:2.

As used herein, the terms “nucleic acid” and “polynucleotide” refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine, and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

An “isolated” nucleic acid molecule is one that is substantially separated from other nucleic acid molecules, which are present in the natural source of the nucleic acid (i.e., sequences encoding other polypeptides). Preferably, an “isolated” nucleic acid is free of some of the sequences, which naturally flank the nucleic acid (i.e. sequences located at the 5′ and 3′ ends of the nucleic acid) in its naturally occurring replicon. For example, a cloned nucleic acid is considered isolated. In various embodiments, the isolated nucleic acid molecule can contain less than about 5 kb, 4 kb, 3 kb, 2 kb, 1 kb, 0.5 kb, or 0.1 kb of nucleotide sequences which naturally flank the nucleic acid molecule in genomic DNA of the cell from which the nucleic acid is derived. A nucleic acid is also considered isolated if it has been altered by human intervention, or placed in a locus or location that is not its natural site, or if it is introduced into a cell by transfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

Specifically excluded from the definition of “isolated nucleic acids” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified nucleic acid makes up less than 5% of the number of nucleic acid inserts in the vector molecules. Further specifically excluded are whole cell genomic DNA or whole cell RNA preparations (including whole cell preparations that are mechanically sheared or enzymatically digested). Even further specifically excluded are the whole cell preparations found as either an in vitro preparation or as a heterogeneous mixture separated by electrophoresis wherein the nucleic acid of the invention has not further been separated from the heterologous nucleic acids in the electrophoresis medium (e.g., further separating by excising a single band from a heterogeneous band population in an agarose gel or nylon blot).

Nucleic acid molecules can be isolated using standard molecular biology techniques and the sequence information provided herein. For example, mRNA can be isolated from a cell, and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed. A nucleic acid molecule can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a known nucleotide sequence can be prepared by standard synthetic techniques, e.g., using an automated DNA synthesizer.

In addition to fragments and fusion polypeptides of the nucleic acid molecules, the present invention includes homologs and analogs of naturally occurring polypeptides. “Homologs” are defined herein as two nucleic acids or polypeptides that have similar, or “identical,” nucleotide or amino acid sequences, respectively. Homologs include allelic variants, orthologs, paralogs, agonists, and antagonists of naturally occurring nucleic acids as defined hereafter. The term “homolog” further encompasses nucleic acid molecules that differ from the determined nucleotide sequence due to degeneracy of the genetic code and thus encode the same polypeptide. As used herein, a “naturally occurring” polypeptide refers to an amino acid sequence that occurs in nature.

An agonist of a polypeptide can retain substantially the same, or a subset, of the biological activities of the polypeptide. An antagonist of a polypeptide can inhibit one or more of the activities of the naturally occurring form of the polypeptide.

Nucleic acid molecules corresponding to natural allelic variants and analogs, orthologs, and paralogs of a nucleic acid sequence can be isolated based on their identity to the known nucleic acids, or a portion thereof, as a hybridization probe according to standard hybridization techniques under stringent hybridization conditions. In an alternative embodiment, homologs of the nucleic acid sequence can be identified by screening combinatorial libraries of mutants, e.g., truncation mutants, for agonist or antagonist activity.

Procedures for introducing a nucleic acid into a cell are well known to those of ordinary skill in the art, and include, without limitation, transfection, transformation or transduction, electroporation, particle bombardment, and the like. In certain embodiments, the nucleic acid is incorporated into a vector or expression cassette that is then introduced into the cell. Other suitable methods for introducing nucleic acids into host cells can be found in Sambrook, et al., Molecular Cloning: A Laboratory Manual. 2nd Ed., Cold Spring Harbor Laboratory, Cold Spring Harbor Laboratory Press, Cold Spring Harbor, N.Y., 1989, and other laboratory manuals such as Methods in Molecular Biology, 1995, Vol. 44, Ed: Gartland and Davey, Humana Press, Totowa, N.J.

As used herein, the term polypeptide refers to a chain of at least four amino acids joined by peptide bonds. The chain may be linear, branched, circular or combinations thereof. The terms “peptide,” “polypeptide,” and “protein” are used interchangeably herein. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

The invention also provides chimeric polypeptides. As used herein, a “chimeric polypeptide” or comprises at least a portion of a member of the reference polypeptide operatively linked to a second, different polypeptide. The second polypeptide has an amino acid sequence corresponding to a polypeptide which is not substantially identical to the reference polypeptide, and which is derived from the same or a different organism. With respect to the chimeric polypeptide, the term “operatively linked” is intended to indicate that the reference polypeptide and the second polypeptide are fused to each other so that both sequences fulfill the proposed function attributed to the sequence used. The second polypeptide can be fused to the N-terminus or C-terminus of the reference polypeptide. For example, in one embodiment, the chimeric polypeptide is a TNFR1-RANK fusion polypeptide in which the TNFR1 extracellular domain is linked to the transmembrane and intracellular domains of RANK.

To determine the percent sequence identity of two amino acid sequences, the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence, then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). Preferably, the isolated amino acid homologs are at least about 50-60%, preferably at least about 60-70%, and more preferably at least about 70-75%, 75-80%, 80-85%, 85-90%, or 90-95%, and most preferably at least about 96%, 97%, 98%, 99%, or more identical.

For the purposes of the invention, the percent sequence identity between two nucleic acid or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

As used herein with regard to hybridization for DNA to a DNA blot, the term “stringent conditions” may refer to hybridization overnight at 60° C. in 10×Denhardt's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. In a preferred embodiment, the phrase “stringent conditions” refers to hybridization in a 6×SSC solution at 6°5C. As also used herein, “highly stringent conditions” refers to hybridization overnight at 65° C. in 10×Denhardt's solution, 6×SSC, 0.5% SDS, and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth & Wahl, (1984) Anal. Biochem. 138:267-284; Current Protocols in Molecular Biology, Chapter 2, Ausubel et al. Eds., Greene Publishing and Wiley-Interscience, New York, 1995; and Tijssen, (1993) Laboratory Techniques in Biochemistry and Molecular Biology: Hybridization with Nucleic Acid Probes, Part I, Chapter 2, Elsevier, N.Y., 1993.

Using the above-described methods, and others known to those of skill in the art, one of ordinary skill in the art can isolate homologs of known nucleic acid sequences. One subset of these homologs is allelic variants. As used herein, the term “allelic variant” refers to a nucleotide sequence containing polymorphisms that lead to changes in the amino acid sequences and that exist within a natural population. Such natural allelic variations can typically result in 1-5% variance in a nucleic acid.

Moreover, nucleic acid molecules encoding a polypeptide from the same or other species such as analogs, orthologs, and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, et al., (1997) Science 278(5338):631-637).

In addition to naturally-occurring variants of a sequence that may exist in the population, the skilled artisan will further appreciate that changes can be introduced by mutation into a nucleotide sequence, thereby leading to changes in the amino acid sequence of the encoded protein, without altering the functional activity of the molecule. For example, nucleotide substitutions leading to amino acid substitutions at “non-essential” amino acid residues can be made in a sequence. A “non-essential” amino acid residue is a residue that can be altered from the wild-type sequence without altering the activity of said protein, whereas an “essential” amino acid residue is required for the activity. Other amino acid residues, however, (e.g., those that are not conserved or only semi-conserved in a domain having biological activity) may not be essential for activity and thus are likely to be amenable to alteration without altering activity. As used herein, the term “mutation” includes substitutions, additions, and deletions of nucleotides or amino acids. One or more amino acid substitutions, additions, or deletions can be introduced into the encoded polypeptide by mutating the nucleic acid using standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Thus, a predicted nonessential amino acid residue is preferably replaced with another amino acid residue from the same side chain family. Alternatively, in another embodiment, mutations can be introduced randomly along all or part of a coding sequence, such as by saturation mutagenesis, and the resultant mutants can be screened for biological activity described herein to identify mutants that retain or do not retain specific biological activity

Antisense polynucleotides are thought to inhibit gene expression of a target polynucleotide by specifically binding the target polynucleotide and interfering with transcription, splicing, transport, translation, and/or stability of the target polynucleotide. Methods are described in the prior art for targeting the antisense polynucleotide to the chromosomal DNA, to a primary RNA transcript, or to a processed mRNA. Preferably, the target regions include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity with the targeted polypeptide sequence.

The antisense nucleic acid can be complementary to an entire coding strand, or to only a portion thereof. In one embodiment, an antisense nucleic acid molecule is antisense to a “coding region” of the coding strand of a nucleotide sequence. The term “coding region” refers to the region of the nucleotide sequence comprising codons that are translated into amino acid residues. In another embodiment, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, but more preferably is an oligonucleotide that is antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al, (1987) Nucleic Acids. Res. 15:6625-6641). The antisense nucleic acid molecule can also comprise a 2′-o-methylribonucleotide (Inoue et al., (1987) Nucleic Acids Res. 15:6131-6148) or a chimeric RNA-DNA analogue (Inoue et al., (1987) FEBS Lett. 215:327-330).

The antisense nucleic acid molecules of the invention are typically administered to a cell or generated in situ such that they hybridize with or bind to cellular mRNA and/or genomic DNA to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

The present invention further provides compositions for RNA interference. In this technique, double-stranded RNA or dsRNA derived from the gene to be analyzed is introduced into the target cell. As used herein, “dsRNA” refers to RNA that is partially or completely double stranded. The dsRNA may have a single stranded overhang at either or both ends of the molecule. This dsRNA is processed into relatively small fragments and can subsequently become distributed throughout the cell. The dsRNA fragments interact, in a cell, with the corresponding endogenously produced messenger RNA, resulting in the endogenous transcript being specifically broken down (Zamore et al., (2000) Cell 101:25-33). This process leads to a loss-of-function mutation having a phenotype that, over the period of a generation, may come to closely resemble the phenotype arising from a complete or partial deletion of the target gene. The invention provides for a composition comprising a dsRNA that is substantially identical to a portion of a target gene of the target cell genome. In certain embodiments of the foregoing, the target gene is selected from the group consisting of (a) the polynucleotide sequence encoding RANK, and (b) a polynucleotide that hybridizes under stringent conditions to a polynucleotide as defined in (a). The polynucleotide and polypeptide sequences encoding mouse RANK are available at GeneID number 21934. The polynucleotide and polypeptide sequences encoding human RANK are available at GeneID number 8792. In other embodiments of the foregoing, the target nucleic acid sequence is identified at GenBank accession number BC080287, NM_{—}008992, BX088552, BC082298, or BC003220.

The invention further provides for a composition comprising a dsRNA consisting of (a) a first stand comprising a sequence substantially identical to 19-49 consecutive nucleotides of the polynucleotide sequence encoding RANK; and (b) a second strand comprising a sequence substantially complementary to the first strand. In certain embodiments, the dsRNA consists of (a) a first stand comprising a sequence substantially identical to 19-49 consecutive nucleotides of the polynucleotide sequence encoding RANK, wherein the nucleotides encode one or more amino acids corresponding to amino acid residues 535-538 of SEQ ID NO:2; and (b) a second strand comprising a sequence substantially complementary to the first strand. Preferably, the dsRNA inhibits expression of a protein encoded by a polynucleotide hybridizing under stringent conditions to the polynucleotide sequence encoding RANK. In further embodiments, the dsRNA has a single stranded overhang at either or both ends. The invention provides for a nucleic acid molecule comprising a regulatory sequence operatively linked to a nucleotide sequence that is a template for one or both strands of the claimed dsRNA. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 basepairs, and wherein after transcription, the RNA transcript folds on itself to form a hairpin.

As an alternative to antisense polynucleotides, ribozymes, sense polynucleotides, or double stranded RNA (dsRNA) can be used to reduce expression of a polypeptide. As used herein, the term “ribozyme” refers to a catalytic RNA-based enzyme with ribonuclease activity that is capable of cleaving a single-stranded nucleic acid, such as an mRNA, to which it has a complementary region. Ribozymes (e.g., hammerhead ribozymes described in Haselhoff & Gerlach, (1988) Nature 334:585-591) can be used to catalytically cleave mRNA transcripts to thereby inhibit translation. A ribozyme having specificity for a nucleic acid can be designed based upon the nucleotide sequence of the cDNA or on the basis of a heterologous sequence to be isolated according to methods taught in this invention. In preferred embodiments, the ribozyme will contain a portion having at least 7, 8, 9, 10, 12, 14, 16, 18, or 20 nucleotides, and more preferably 7 or 8 nucleotides, that have 100% complementarity to a portion of the target RNA. In one embodiment, the ribozyme target comprises nucleotides encoding one or more amino acids corresponding to amino acid residues 535-538 of SEQ ID NO:2. Methods for making ribozymes are known to those skilled in the art. See, e.g., U.S. Pat. Nos. 6,025,167; 5,773,260; and 5,496,698.

The term “dsRNA,” as used herein, refers to RNA hybrids comprising two strands of RNA. The dsRNAs can be linear or circular in structure. The hybridizing RNAs may be substantially or completely complementary. By “substantially complementary,” is meant that when the two hybridizing RNAs are optimally aligned using the BLAST program as described above, the hybridizing portions are at least 95% complementary. Preferably, the dsRNA will be at least 100 base pairs in length. Typically, the hybridizing RNAs will be of identical length with no over hanging 5′ or 3′ ends and no gaps. However, dsRNAs having 5′ or 3′ overhangs of up to 100 nucleotides may be used in the methods of the invention.

The dsRNA may comprise ribonucleotides, ribonucleotide analogs such as 2′-O-methyl ribosyl residues, or combinations thereof. See, e.g., U.S. Pat. Nos. 4,130,641 and 4,024,222. A dsRNA polyriboinosinic acid:polyribocytidylic acid is described in U.S. Pat. No. 4,283,393. Methods for making and using dsRNA are known in the art.

A useful method to ascertain the level of transcription of the gene (an indicator of the amount of mRNA available for translation to the gene product) is to perform a Northern blot (For reference, see, for example, Ausubel et al., (1988) Current Protocols in Molecular Biology, Wiley: New York). The information from a Northern blot at least partially demonstrates the degree of transcription of the transformed gene. Total cellular RNA can be prepared from cells, tissues, or organs by several methods, all well-known in the art, such as that described in Bormann, et al., (1992) Mol. Microbiol. 6:317-326. To assess the presence or relative quantity of polypeptide translated from this mRNA, standard techniques, such as a Western blot, may be employed. These techniques are well known to one of ordinary skill in the art. (See, for example, Ausubel et al., (1988) Current Protocols in Molecular Biology, Wiley: New York).

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Liposomally-encapsulated expression vectors can also be used for gene delivery. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., retroviral, lentiviral, adenoviral, adeno-associated viral (AAV), herpes viral, alphavirus, astrovirus, coronavirus, orthomyxovirus, papovavirus, paramyxovirus, parvovirus, picornavirus, poxvirus, or togavirus vectors), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. As used herein with respect to a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990), including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides.

Another aspect of the invention pertains to isolated polypeptides, and biologically active portions thereof. An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations in which the polypeptide is separated from some of the cellular components of the cells in which it is naturally or recombinantly produced. In one embodiment, the language “substantially free of cellular material” includes preparations of a polypeptide having less than about 30% (by dry weight) of a contaminating polypeptide, more preferably less than about 20% of a contaminating polypeptide, still more preferably less than about 10% of a contaminating polypeptide, and most preferably less than about 5% a contaminating polypeptide.

When the polypeptide or biologically active portion thereof is recombinantly produced, it is also preferably substantially free of culture medium, i.e., culture medium represents less than about 20%, more preferably less than about 10%, and most preferably less than about 5% of the volume of the polypeptide preparation. The language “substantially free of chemical precursors or other chemicals” includes preparations in which the polypeptide is separated from chemical precursors or other chemicals that are involved in the synthesis of the polypeptide.

The present invention also provides antibodies that specifically bind to a polypeptide, or a portion thereof, as encoded by a nucleic acid described herein. Antibodies can be made by many well-known methods (See, e.g., Harlow and Lane, “Antibodies; A Laboratory Manual,” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al., (1992) Bio/Technology 10:163-167; Bebbington et al., (1992) Bio/Technology 10:169-175).

The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane, “Antibodies, A Laboratory Manual” Cold Spring Harbor Publications, New York, (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al, eds., “Basic and Clinical Immunology,” (Lange Medical Publications, Los Altos, Calif., Fourth Edition) and references cited therein, and in Harlow and Lane “Antibodies, A Laboratory Manual” Cold Spring Harbor Publications, New York, 1988.

The invention further encompasses modulation of RANK-mediated signaling through compounds that interact with the novel motif of the invention, corresponding to amino acids 535-538 of SEQ ID NO:2.

Identification of the components of the RANK signaling pathway mediated by the novel motif of the invention is readily determined by one of ordinary skill in the art. For example, components that directly interact with the IVVY (residues 535-538 of SEQ ID NO:2) motif can be isolated using the yeast 2-hybrid system as described herein. In certain embodiments, a component of a RANK-mediated signaling pathway is encoded by a nucleic acid selected from the group consisting of BC080287 (Gene ID number 56353); NM_{—}008992 (Gene ID number 19300), BX088552, BC082298, BC003220 (Gene ID number 13559), and homologs and analogs thereof.

Interactions between the novel motif and the identified components can be evaluated by at least co-immunoprecipitation. Roles of the identified components in the osteoclasteogenic process can be determined, for example, by RNA interference (RNAi) as described above. Candidate molecules that play a role in the osteoclasteogenic process and/or interact with the novel motif of the invention are useful for detecting modulation of at least one RANK-mediated signaling pathway.

In addition, the activation of RANK-mediated signaling pathways can be evaluated by monitoring osteoclast formation or function. Methods suitable for this purpose include, but are not limited to, osteoclastogenesis or bone resportion assays. See, for example, Armstrong et al., (2002) J. Biol. Chem., 277:44347-44356 and Ye et al., (2002) Nature, 418:443-44. A typical osteoclastogenesis assay includes introducing a polypeptide of the present invention into an osteoclast precursor cell, such as a bone marrow macrophage or a splenic hematopoietic progenitor cell, followed by adding a ligand to induce oligomerization of the polypeptide, thereby initiating cellular differentiation. Compounds capable of inhibiting or interfering with osteoclast differentiation can be identified by comparing the level of osteoclastogenesis in the presence of the compound to that in the absence of the compound. In many cases, a compound thus identified can reduce osteoclast differentiation or osteoclastogenesis by at least approximately 30%, 40%, 50%, 60%, 70%, 80%, 90% or 100%.

Osteoclast bone resorption assays can also be used to evaluate activities of RANK signaling pathways mediated by the novel motif of the invention. RANK modulators capable of inhibiting osteoclast bone resorption can be identified by comparing the level of bone resorption in the presence of the modulators to that in the absence of the modulators. In many cases, a modulator thus identified can inhibit osteoclast-dependent bone resorption activities by at least approximately 30%, 40%, 50%, 60%, 70%, 80%, 90% or 100%.

Any detection methodology known in the art may be used to assess interactions between the novel motif of the invention and substrates that interact with RANK. These methodologies include, but are not limited to, surface plasmon resonance (e.g., Biacore), radioimmune based assays, and fluorescence polarization binding assays. When performed in the presence of a test compound, the ability of the test compound to modulate (e.g., inhibit or enhance) the protein-protein binding affinity is determined. For example, either the novel motif and substrates that interact with the motif can be labeled with a detectable moiety so that the binding can be measured and the effectiveness of various inhibitors or enhancers judged. The detectable moiety allows for detection by direct or indirect means. Direct means include, but are not limited to luminescence, chemiluminescence, fluorescence, radioactivity, optical or electron density. Indirect means include but are not limited to an enzyme or epitope tag.

A detectable moiety can be a compound or molecule that is distinguishable from the surroundings. The art is replete with examples of detectable moieties that can be used in screening assays. In the present specification, the term “label” is used interchangeably with “detectable moiety.” For example, detectable moieties may be any moiety based on luminescence, chemiluminescence, fluorescence, radioactivity, enzymatic reactions, calorimetric, optical or electron density. It is to be understood that the screening assays described herein for identifying test compounds that modulate the protein:protein interaction may employ one or more of the detectable moieties known in the art. The protein can be directly or indirectly labeled with a detectable moiety. Such moieties can be attached or labeled to the protein by any suitable conventional procedure. For instance, the functional groups on amino acid side chains can be reacted with functional groups on a desired moiety to form covalent bonds. Alternatively, the protein can be derivatized to generate or attach a desired reactive functional group. The derivatization can involve attachment of one or more linkers or couplers, such as any of the family of bifunctional coupling reagents available for attaching various molecules to polypeptides (Pierce Chemical Company, Rockford, Ill.).

In many embodiments, homogeneous assay formats are used to determine interactions between polypeptides, such as fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence. In another aspect, the inventive methods utilize heterogeneous assay formats such as enzyme-linked immunosorbant assays (ELISA) or radioimmunoassays.

One such assay is based on fluorescence resonance energy transfer (FRET) between two fluorescent labels, an energy donating long-lived chelate label and a short-lived organic acceptor. The energy transfer occurs when the two labels are brought in close proximity via the molecular interaction between the novel motif of the invention and downstream signaling molecules.

Another useful assay is a bioluminescence resonance energy transfer (BRET), such as that described in Xu et al., (1999) PROC. NATL. ACAD. SCI. USA, 96:151. Similar to a FRET assay, BRET is based on energy transfer from a bioluminescent donor to a fluorescent acceptor protein. However, a green fluorescent protein (GFP) is used as the acceptor molecule, eliminating the need for an excitation light source. Exemplary BRET assays include BRET and BRET² from Packard BioScience (Meriden, Conn.). It is understood that the novel motif of the invention and downstream signaling molecule may be configured in the assay in any workable manner, such as alternatively labeling either polypeptide with GFP. It is further understood that inhibitors and enhancers of the polypeptide interaction may be identified.

DELFIA® (dissociated enhanced lanthanide fluoroimmunoassay) is a solid-phase assay based on time-resolved fluorometry analysis of lanthanide chelates (see, for example, U.S. Pat. No. 4,565,790). For this type of assay, microwell plates are coated with a first protein. The binding partner is conjugated to europium chelate or cryptate, and added to the plates. After suitable incubation, the plates are washed and a solution is added to dissociate europium ions from solid phase bound protein into solution, thereby forming highly fluorescent chelates with ligands present in the solution, after which the plates are read using a plate reader to detect emission at 615 nm.

Another assay that may be employed is a FlashPlate® (Packard Instrument Company, IL) based assay. This assay measures the ability of compounds to inhibit protein-protein interactions. FlashPlates are coated with a first protein, then washed to remove excess protein. For the assay, compounds to be tested are incubated with the second protein, and I¹²⁵ labeled antibody against the second protein is added to the plates. After suitable incubation and washing, the amount of radioactivity bound is measured using a scintillation counter.

Further embodiments include the AlphaScreen™ assay (Packard Instrument Company, Meriden, Conn.). AlphaScreen technology is an “Amplified Luminescent Proximity Homogeneous Assay” method utilizing latex microbeads (250 nm diameter) containing a photosensitizer (donor beads), or chemiluminescent groups and fluorescent acceptor molecules (acceptor beads). Upon illumination with laser light at 680 nm, the photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity to the donor bead (i.e., by virtue of the interaction of two polypeptides), the singlet-state oxygen molecules reacts with chemiluminescent groups in the acceptor beads, which immediately transfer energy to fluorescent acceptors in the same bead. These fluorescent acceptors shift the emission wavelength to 520-620 nm, resulting in a detectable signal. Inhibitors of the interaction of the polypeptides will thus reduce the shift in emission wavelength, whereas enhancers of this interaction would increase it.

In one specific embodiment, a screening method of the present invention comprises the steps of forming a composition comprising the novel motif of the invention, a downstream signaling molecule, and the test compound; assaying for the level of interaction of the two polypeptides; and comparing the level obtained in the presence of the test compound to that obtained in the absence of the test compound, such that if the level obtained differs, a compound that affects the interaction of the two polypeptides, and thus of a RANK-mediated signaling pathway, is identified. Preferably, at least one of the two polypeptides can be labeled with a detectable moiety. One of the polypeptides can be soluble, and the other can be bound, although alternative assay formats are possible and well known. The test compound can be added to the composition after addition of the two polypeptides, before both polypeptides are added, or after one polypeptide is added and before the other is added. The interaction of the polypeptides that may be influenced by the test compound includes reciprocal binding of the polypeptides. For example, a test compound may partially or completely inhibit binding of the novel motif of the invention to the downstream signaling polypeptide. This partial or complete inhibition of binding can be measured in various ways, such as determining the binding constant in the presence and absence of the test compound. In other embodiments, the binding affinity and/or binding avidity between the polypeptides may be measured with and without the test compound.

Any of the above-described methods can be incorporated in high throughput test systems so that large numbers of test molecules can be screened within a short amount of time. The assays can be performed in a variety of formats, including protein-protein binding assays, biochemical screening assays, immunoassays, cell based assays, etc. These assay formats are well known in the art. The screening assays of the present invention are amenable to screening of chemical libraries and are suitable for the identification of small molecule drug candidates, antibodies, peptides, peptidomimetics, and the like. Chemical libraries include commercially available combinatorial chemistry compound libraries from companies such as, but not limited to, Sigma-Aldrich (St. Louis, Mo.), Arqule (Woburn, Mass.), Enzymed (Iowa City, Iowa), Maybridge Chemical Co. (Trevillett, Cornwall, UK), MDS Panlabs (Bothell, Wash.), Pharmacopeia (Princeton, N.J.), and Trega (San Diego, Calif.).

Moreover, combinations of screening assays can be used to find molecules that regulate the biological activity of RANKL interactions. In using combinations of various assays to screen for test compounds, it is understood that any of the assays described herein may be used in any order and combination. For example, one embodiment may comprise first determining whether a test compound binds to RANK or modulates the binding between RANK and a downstream signaling molecule by using an assay that is amenable to high throughput screening. Test compounds identified in this manner are then added to a biological assay to determine biological effects. By observing the effect that test compounds have on the interaction between RANK and a downstream signaling molecule in various binding assays, on RANK-mediated activity in biological function tests, or in cell based screens, compounds that are potential therapeutics because they can modulate the interaction between RANK and a downstream signaling molecule are identified. These compounds will be useful in treating or preventing disease or conditions with which RANK-mediated signaling is implicated.

RANK modulators can also be identified based on rational drug design. One goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules. In one approach, one would generate a three-dimensional structure for RANK or a downstream signaling molecule of RANK. This could be accomplished by x-ray crystallograph, NMR, computer modeling, or by a combination of these approaches. An alternative approach, “alanine scan,” involves the random replacement of residues throughout molecule with alanine, and the resulting affect on function determined.

It also is possible to isolate a RANK or downstream signaling molecule specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using any method suitable for producing antibodies, using an antibody as the antigen.

In many cases, an inhibitor identified by the present invention can inhibit RANK-downstream molecule binding or consequential biological activity (e.g., osteoclastogenesis or bone resorption) by at least 30%, 40%, 50%, 60%, 70%, 80%, 90% or more. Similarly, a stimulator of the present invention can increase the RANK-downstream molecule binding or consequential biological activity by at least 20%; 30%, 40%, 50% or more. Those of ordinary kill in the art will recognize that RANK modulators with different levels of inhibition or enhancement may be useful for different applications (e.g., for treatment of different disease states).

RANK modulators of the present invention can be any type of molecule, such as small molecules, peptide, peptide mimics, or antibodies. Exemplary antibodies amenable to the present invention include, but are not limited to, monoclonal antibodies, mono-specific antibodies, poly-specific antibodies, non-specific antibodies, humanized antibodies, human antibodies, single-chain antibodies, chimeric antibodies, synthetic antibodies, recombinant antibodies, hybrid antibodies, Fab, F(ab′)₂, Fv, scFv, Fd, dAb, or biologically active fragments thereof. In one embodiment, an antibody of the present invention includes two or more antigen-binding sites, each of which recognizes a different respective motif. In one example, the binding affinity for the motif is at least 10⁻⁵ M⁻¹, 10⁻⁶ M⁻¹, 10⁻⁷ M⁻¹, 10⁻⁸ M⁻¹, 10⁻⁹ M⁻¹, or stronger.

In one embodiment, the target cells are contacted with an effective amount of a modulator of the RANK-mediated signaling pathway. As used herein, the term “effective amount” of a modulator of the RANK-mediated signaling pathway refers to that concentration of the compound that is sufficient to affect differentiation of a target cell towards a desired cell lineage, preferably, towards or away from an osteoclast lineage. The desired concentration is readily determined by one of ordinary skill in the art.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” is used to refer to the physical selection of one or more cells out of a group of two or more cells, wherein the cells are selected based on cell morphology and/or the expression of various markers.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “contacting” (i.e., contacting a cell e.g. a target cell, with a compound) is intended to include incubating the compound and the cell together in vitro (e.g., adding the compound to cells in culture). The term “contacting” is not intended to include the in vivo exposure of cells to a modulator of the RANK-mediated signaling pathway that may occur naturally in a subject (i.e., exposure that may occur as a result of a natural physiological process). The step of contacting the cell with a test compound can be conducted in any suitable manner.

The compositions and methods described herein have several useful features. For example, the compositions and methods described herein are useful for modeling the stages of bone development. Furthermore, the compositions and methods described herein can also serve for therapeutic intervention in disease states, such as osteoporosis, osteopenia, or other bone-loss or bone density decreasing disorders. For example, compounds that inhibit the activity of RANK can be formulated into a pharmaceutical formulation for the treatment of a disease state, such as, but not limited to osteoporosis, osteopenia, or other bone-loss or bone density decreasing disorders.

The cell types that differentiate from osteoclast precursor cells after contact with a modulator of the RANK-mediated signaling pathway have several uses in various fields of research and development including but not limited to drug discovery, drug development and testing, toxicology, production of cells for therapeutic purposes as well as basic science research. These cell types express molecules that are of interest in a wide range of research fields. These include the molecules known to be required for the functioning of the various cell types as described in standard reference texts. These molecules include, but are not limited to, cytokines, growth factors, cytokine receptors, extracellular matrix, transcription factors, secreted polypeptides and other molecules, and growth factor receptors. In addition, the cells can be used as a source of nuclear material for nuclear transfer techniques and used to produce cells, tissues or components of organs for transplant. The test compounds that decrease differentiation of osteoclasts also have a number of functions, including but not limited to the treatment of various bone disorders, usefulness in determining the molecular signaling pathways involved in bone development, demineralization and bone regrowth.

The progression of the target cell culture to the desired cell lineage or response to a test compound can be monitored by quantitating expression of marker genes characteristic of the desired cell lineage as well as the lack of expression of marker genes characteristic of osteoclast progenitor cells and other cell types. One method of quantitating gene expression of such marker genes is through the use of quantitative PCR (Q-PCR). Methods of performing Q-PCR are well known in the art. Other methods that are known in the art can also be used to quantitate marker gene expression. Marker gene expression can be detected by using antibodies specific for the marker gene of interest.

In some embodiments of the present invention, cells of the desired cell lineage can be isolated by using an affinity tag that is specific for such cells. One example of an affinity tag specific for a target cell is an antibody that is specific to a marker polypeptide that is present on the cell surface of the target cell but which is not substantially present on other cell types that would be found in a cell culture produced by the methods described herein.

As described herein, the invention encompasses a method of improving bone mass in an individual having a bone-related disorder, by administering to the individual a therapeutically effective amount of a compound. As used herein, the phrase “bone-related disorder” refers to a disorder wherein bone formation, deposition, or resorption is abnormal. Bone-related disorders include, but are not limited to, osteoporosis, bone fractures, hypercalcemia of malignancy, osteopenia or osteolytic lesions due to bone metastases, periprosthetic osteolysis, familial expansile osteolysis, periodontal disease, tooth loss, rheumatoid arthritis, osteoarthritis, hyperparathyroidism, Paget's disease, osteodystrophy, myositis ossificans, Bechterew's disease, malignant hypercalcernia, bone loss, bone abnormalities due to steroid hormone treatment, bone abnormalities caused by cancer therapeutics, abnormally increased bone turnover, osteomalacia, Bechet's disease, hyperostosis, osteopetrosis, osteogenesis imperfecta, rachitis, immobilization-induced osteopenia, expansile skeletal hyperphosphatasia, and glucocorticoid-induced osteoporosis.

Another aspect of this invention is directed to methods for strengthening a bone graft, inducing vertebral synostosis, enhancing long bone extension, the treatment and promotion of healing of bone fractures and osteotomies, enhancing bone healing following facial reconstruction, maxillary reconstruction and/or mandibular reconstruction in a vertebrate, e.g., a mammal (including a human being), comprising administering to said vertebrate a therapeutically effective amount of a compound of the current invention, a prodrug or a pharmaceutically acceptable salt thereof, or a stereoisomer or diastereomeric mixture of said compound, prodrug or salt. The composition may be applied locally to the site of bone reconstruction or may be administered systemically.

Administration of the compounds of this invention can be via any mode that delivers the compound systemically and/or locally (e.g., at the site of the bone fracture, osteotomy, or orthopedic surgery).

In the methods of the present invention, the compounds described herein and determined using the screening methods described herein, can form the active ingredient, and are typically administered in admixture with suitable pharmaceutically acceptable diluents, excipients, adjuvants or carriers (collectively referred to herein as “carrier” materials) suitably selected with respect to the intended form of administration, that is, oral tablets, capsules, pills, powders, granules, elixirs, tinctures, suspensions, syrups and the like, and consistent with conventional pharmaceutical practices. Likewise, they may also be administered in intravenous (bolus or infusion), intraperitoneal, intranasal, rectal, topical, subcutaneous, intramuscular or transdermal form, all using forms well known to those of ordinary skill in the pharmaceutical arts.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

The compounds can be applied to the sites of bone fractures or osteotomies, for example, either by injection of the compound in a suitable solvent (e.g., an oily solvent such as arachis oil) to the cartilage growth plate or, in cases of open surgery, by local application thereto of the compound in a suitable vehicle, carrier or diluent such as bone-wax, demineralized bone powder, polymeric bone cements, bone sealants, etc. Alternatively, local application can be achieved by applying a solution or dispersion of the compound in a suitable carrier or diluent onto the surface of, or incorporating it into solid or semi-solid implants conventionally used in orthopedic surgery, such as dacron-mesh, gel-foam and kiel bone, or prostheses.

As used herein, the phrase “pharmaceutically acceptable” refers to an agent that does not interfere with the effectiveness of the biological activity of an active ingredient, and which may be approved by a regulatory agency of the Federal government or a state government, or is listed in the U.S. Pharmacopeia or other generally recognized pharmacopeia for use in animals, and more particularly for use in humans. Accordingly, suitable pharmaceutically acceptable carriers include agents that do not interfere with the effectiveness of a pharmaceutical composition.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Methods of preparing various pharmaceutical compositions with a certain amount of active ingredient are known to those skilled in the art. For examples of methods of preparing pharmaceutical compositions, see Remington: The Science and Practice of Pharmacy, Mack Publishing Company, Easton, Pa., 19th Edition (1995).

The instant compounds are also useful in combination with known agents useful for treating bone-related disorders. Combinations of the presently disclosed compounds with other agents useful in treating osteoporosis or other bone-related disorders are within the scope of the invention. A person of ordinary skill in the art would be able to discern which combinations of agents would be useful based on the particular characteristics of the drugs and the disease involved. Such agents include but are not limited to the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; selective serotonin reuptake inhibitors (SSRIs); and the pharmaceutically acceptable salts and mixtures thereof.

The term “administration” and variants thereof (e.g., “administering” a compound) in reference to a compound of the invention means introducing the compound or a prodrug of the compound into the system of the individual in need of treatment. When a compound of the invention or prodrug thereof is provided in combination with one or more other active agents (e.g., a bisphosphonate, etc.), “administration” and its variants are each understood to include concurrent and sequential introduction of the compound or prodrug thereof and other agents.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

When a compound according to this invention is administered into a human subject, the daily dosage will normally be determined by the prescribing physician with the dosage generally varying according to the age, sex, weight, and response of the individual patient, as well as the severity of the patient's symptoms, the route of administration; and the particular compound or salt thereof employed. An ordinarily skilled physician, veterinarian or clinician can readily determine and prescribe the effective amount of the drug required to prevent, counter or arrest the progress of the condition.

In one exemplary application, a suitable amount of compound is administered to a mammal undergoing treatment. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three, four, or more times daily. The doses can be administered at intervals such as once daily, once weekly, or once monthly. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

Toxicity and therapeutic efficacy of a RANK modulator can be determined by standard pharmaceutical procedures in cell culture or experimental animal models. For instance, the LD₅₀ (the dose lethal to 50% of the population) and the ED₅₀ (the dose therapeutically effective in 50% of the population) can be determined. The dose ratio between toxic and therapeutic effects is the therapeutic index, and can be expressed as the ratio LD₅₀/ED₅₀. In many cases, RANK modulators that exhibit large therapeutic indices are selected.

The data obtained from cell culture assays and animal studies can be used in formulating a range of dosages for use in humans. In one embodiment, the dosage lies within a range of circulating concentrations that exhibit an ED₅₀ with little or no toxicity. The dosage may vary within this range depending upon the dosage form employed and the route of administration utilized.

The compositions and methods of the present invention are administered and carried out until the desired therapeutic effect is achieved. The term “until the desired therapeutic effect is achieved”, as used herein, means that the therapeutic agent or agents are continuously administered, according to the dosing schedule chosen, up to the time that the clinical or medical effect sought for the disease or condition being treated is observed by the clinician or researcher. For methods of treatment of the present invention, the pharmaceutical composition is continuously administered until the desired improvement in bone mass or structure is observed. In such instances, achieving an improvement in bone mass or a replacement of abnormal bone structure with normal bone structure are the desired objectives. For methods of prevention of the present invention, the pharmaceutical composition is continuously administered for as long as necessary to prevent the undesired condition. In such instances, maintenance of bone mass density is often the objective. Progress of a treatment can be monitored by periodic assessment of disease progression. The progress can be monitored, for example, by X-rays, MRI or other imaging modalities, synovial fluid analysis, or clinical examination. Non-limiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treat”, “treating”, or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

As used herein, the term “improving” with respect to bone mass includes increasing or maintaining the current bone mass of an individual, and includes slowing the rate of bone loss. As such, the term reducing or inhibiting the resorption of bone in bone-related disorders. As described herein, determining the modulation of a RANK-mediated signaling pathway, or a modulation of osteoclast formation in vitro contact with a compound is predictive that the compound is useful for treating a bone-related disorder, or improving bone mass.

The term “bone resorption,” as used herein, refers to the process by which osteoclasts degrade bone.

As used herein, the term “bone mass” refers to bone mass per unit area, which is sometimes referred to as bone mineral density.

In the present invention, in one aspect, the compounds can be used to inhibit bone resorption, or more specifically to inhibit undesired or abnormal bone resorption. The term “abnormal bone resorption”, as used herein means a degree of bone resorption that exceeds the degree of bone formation, either locally, or in the skeleton as a whole. Alternatively, “abnormal bone resorption” can be associated with the formation of bone having an abnormal structure, as in Paget's disease. In another aspect, the compounds can be used to promote bone resorption, or more specifically to resorb undesired or abnormal bone formation. The term “abnormal bone formation”, as used herein means a degree of bone formation that exceeds the degree of bone resorption, either locally, or in the skeleton as a whole.

The term “bone resorption inhibiting”, as used herein, means preventing bone resorption by the direct or indirect alteration of osteoclast formation or activity. Inhibition of bone resorption refers to prevention of bone loss, especially the inhibition of removal of existing bone either from the mineral phase and/or the organic matrix phase, through direct or indirect alteration of osteoclast formation or activity.

As used herein, the term “osteoclast precursor cell” refers to a cell that differentiates towards the osteoclast lineage upon treatment with known osteoclast-promoting agents, such as, dexamethasone, 1,25-dihydroxyvitamin D3, M-CSF, RANKL, TNF-α, IL-1 and prostaglandin E2. In certain embodiments, the osteoclast precursor cell is a pre-osteoclast, a bone marrow macrophage (BMM), a peripheral monocyte, a spleen monocyte, or an immortalized mouse macrophage cell line, such as, but not limited to, RAW264.6.

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Methods

### Chemicals and Reagents

Chemicals were purchased from Sigma (St. Louis, Mo.) unless indicated otherwise. Synthetic oligonucleotides were purchased from Sigma-Genosys (The Woodlands, Tex.). Blasticidin was from EMD Biosciences, Inc (San Diego, Calif.). Antibodies against the external domain of mouse TNFR1 (for flow cytometry) (TNF-R1, sc-12746PE), NFATc1 (sc-7294) and c-fos (sc-253), were purchased from Santa Cruz Biotechnology, Inc (Santa Cruz, Calif.). Recombinant mouse TNFα (410-TRNC-050) was from R&D Systems (Minneapolis, Minn.). The following antibodies were purchased from Cell Signaling Technology, Inc (Beverly, Mass.): antibodies against IκBα (#9242), phospho-IκBα (#9241), p44/42ERK (#9102), phospho-p44/42ERK (#9101), JNK (#9252), phospho-JNK (#9251), p38 (#9212), phospho-p38 (#9211), Akt (#9272) and phospho-Akt (#9271).

Construction of TNFR1/RANK Chimeric cDNA

A chimeric cDNA comprising mouse TNFR1 external domain (from AA 1 to AA 210 of SEQ ID NO:1) linked in frame to the transmembrane and cytoplasmic domains of mouse RANK (AA 210 to AA 625 of SEQ ID NO:2) was constructed using standard molecular cloning techniques. The amino acid sequences of mouse TNFR1 and RANK genes have Entrez accession numbers NP_{—}035739 (SEQ ID NO:1) and NP_{—}033425 (SEQ ID NO:2), respectively. cDNA fragment encoding mouse TNFR1 external domain was amplified by RT-PCR using total RNA isolated from mouse BMMs and a pair of primers containing XbaI sites. The TNFR1 cDNA fragment was then subcloned into pBluescript II SK+ cloning vector (Stratagene, La Jolla, Calif.) at XbaI site, resulting in a plasmid named SK-TNFR1. cDNA encoding the RANK transmembrane and cytoplasmic domains was also amplified by RT-PCR using total RNA from mouse BMMs with a forward primer containing SpeI site and a reverse primer containing BamHI site. The RANK cDNA fragment was then subcloned into SK-TNFR1 between SpeI and BamHI, giving rise to a plasmid named SK-TNFR1-RANK. The orientation and sequence of the chimeric cDNA was confirmed by sequencing.

### Construction of Deletion Mutants and Mutagenesis

Construction of internal deletion mutants was performed based on a method described previously (Barnhart, (1999) Biotechniques 26, 624-627). Briefly, to delete a region in the RANK cytoplasmic tail in TNFR1-RANK chimera, the SK-TNFR1-RANK plasmid as described in (Liu et al., (2004) J. Biol. Chem. 279, 54759-54769) and above was used as the template with two primers to perform PCR with Pfu polymerase (high fidelity polymerase). Both primers contain a Mlu I restriction site at their 5′ end and had a sequence complementary to a desired region of the template. The PCR reaction generated a linear full-plasmid length product lacking the desired sequence and the product contained a Alu I site at each of its ends. The PCR product was excised with Alu I, religated, and sequenced to confirm that no mutations were introduced during PCR amplification. Then, the TNFR1-RANK construct containing the internal deletion was excised and subcloned into pMX-puro retrovirus vector.

Mutations were generated in SK-TNFR1-RANK using the QuickChange™ Site-directed Mutagenesis Kit (Stratagene). To generate PM1, PM2, PM3 and PM4, the following primers were used: for PM1, 5′-AACTTCAAGGGTGGACTCATCGTGGT-GTAT-3′ (SEQ ID NO:4) and 5′-TTGAAGTTCCCACTTGAGTAGCACCACATA-3′ (SEQ ID NO:5); for PM2,5′-AAGGGTGACATCCTCGCGGTGTATGTCAGC-3′ (SEQ ID NO:6) and 5′-TTCCCACTGTAGGAGC-GCCACATACAGTCG-3′ (SEQ ID NO:7); for PM3,5′-GACATCATCGTGGCGTTTGTCAGC-CAGACC-3′ (SEQ ID NO:8) and 5′-CTGTAGTAGCACCGCAAACAGTCGGTCTGG-3′ (SEQ ID NO:9); and for PM4: 5′-ATCGTGGTGTATGCCGCCCAGACCTCGCAG-3′ (SEQ ID NO:10) and 5′-TAGCACCACATACGGC-GGGTCTGGAGCGTC-3′ (SEQ ID NO:11). The mutated sites were confirmed by sequencing and other regions in the chimeric cDNA were sequenced to confirm that no mutations were introduced by PCR amplification during the mutagenesis. The mutant chimeric cDNAs were then subcloned into pMX-puro plasmids as described above for virus preparation.

### Preparation of Retrovirus and Infection of Bone Marrow Macrophages (BMM)

The retrovirus vector pMX-puro (Onishi et al., (1998) Mol. Cell. Biol., 18:3871-3879) and the Plat-E packaging cells (Morita et al., (2000) Gene Therapy, 7:1063-1066) were used. The chimeric cDNA (TNFR1-RANK) from SK-TNFR1-RANK was subcloned into pMX-puro to generate plasmid construct named pMX-puro-TNFR1-RANK. Essentially, Plat-E cells were cultured in DMEM with 10% heat-inactivated FBS supplemented with puromycin and blasticidin as previously described (Morita et al., (2000) Gene Therapy 7, 1063-1066). pMX-puro-TNFR1-RANK and its various deletion/point mutation mutants were transiently transfected into Plat-E cells using Lipofectamine Plus reagent (Invitrogen, Carlsbad, Calif.). Virus supernatant was collected at 48, 72 and 96 hours after transfection.

### Culturing and Infection of Bone Marrow Macrophages (BMMs)

Bone marrow cells were isolated from long bones of 4-8 week old TNFR1^{-/-}R2^{-/-} double knockout mice (The Jackson Laboratory, Bar Harbor, Me.) or wild-type mice (Harlan Industries, Indianapolis, Ind.), as described in Feng et al., (2001) J. Clin. Invest., 107:1137-1144. BMMs were prepared by culturing isolated bone marrow cells in α-MEM containing 10% heat-inactivated FBS in the presence of 0.1 volume of culture supernatant of M-CSF-producing cells for 2 days as previously described in Takeshita et al., (2000) J. Bone Miner. Res., 15: 1477-1488. Cells were then infected with virus for 24 hours in the presence of 0.1 volume of culture supernatant of M-CSF-producing cells and 8 μg/ml polybrene. Cells were further cultured in the presence of M-CSF and 2 μg/ml puromycin for selection and expansion of transduced cells. Selected cells were subsequently used for various studies.

### In Vitro Osteoclastogenesis Assays

Retrovirally infected BMMs were cultured in 24-well tissue culture plates (1×10⁵ cells/well) in α-MEM containing 10% heat-inactivated FBS in the presence of 0.01 volume of culture supernatant of M-CSF-producing cells (final M-CSF concentration was 22 ng/ml) and 100 ng of GST-RANKL (Lam et al., (2000) J. Clin. Invest. 106, 1481-1488). In osteoclast formation assays involving the use of the chimeric receptor, TNFα was added at concentrations as indicated in individual assays. Osteoclasts began to form on day 3 and cultures were stained for tartrate-resistant acid phosphatase (TRAP) activity on day 5 using a commercial kit (Sigma, 387-A).

### Bone Resorption Assay

Osteoclasts were generated on bovine cortical bone slices from infected or uninfected BMMs with the stimulation of M-CSF (44 ng/ml) and RANKL (100 ng/ml) for 3 days. After osteoclasts were formed, the cultures were then treated with different factors as indicated in the individual assays and then continued for three more days. Bone slices were then harvested. Cells were removed from the bone slices with 0.25 M ammonium hydroxide and mechanical agitation. The bone slices were then subjected to scanning electron microscopy (SEM).

### Osteoclast Survival Assay

Osteoclasts were generated in tissue culture dishes from infected or uninfected BMMs with the stimulation of M-CSF (44 ng/ml) and RANKL (100 ng/ml) for 3 days. After osteoclasts were formed, the cultures were then treated with different factors as indicated in the individual assays and then continued for six more hours. The cultures were then stained for TRAP activity using a commercial kit according to the manufacturer's instructions (Sigma, 387-A). Osteoclast survival was determined by counting the cells with strong TRAP activity and intact plasma membrane.

### Flow Cytometric Analysis

Retrovirally infected BMMs (up to 1×10⁶ cells) were suspended in 200 μl PBS/Azide. Cells were then blocked with 1 μg 2.4G2 antibody (Unkeless, (1979) J. Exp. Med. 150, 580-596) for 30 minutes on ice. Under dim light, 20 μl of TNFR1 antibody conjugated with phycoerythrin (Santa Cruz, Calif., sc-12746PE) or control IgG was added to the cell suspension and cells were incubated on ice for 30 minutes. Cells were washed twice with 1 ml cold PBS/Azide and resuspended in 300 μl cold PBS/Azide. 200 μl cold 0.5% paraformaldehyde solution was added to fix the cells. Flow cytometric analysis was performed using a Becton-Dickinson FACSan (Becton-Dickinson Immunocytometry Systems, Mountain View, Calif.).

### Western Analysis

BMMs infected with retrovirus or control uninfected BMMs were cultured in serum-free α-MEM in the absence of M-CSF for approximately 16 hours before treatment with RANKL or TNFα for various times as indicated in individual experiments. Cells were washed twice with ice-cold phosphate-buffered saline (PBS) and then lysed in buffer containing 20 mM Tris, pH 7.5, 150 mM NaCl, 1 mM EDTA, 1 mM EGTA, 1% Triton X-100, 2.5 mM sodium pyrophosphate, 1 mM β-glycerophosphate, 1 mM Na₃VO₄, 1 mM NaF, and 1× protease inhibitor cocktail 1 (Sigma, P-2850) and 1× protease inhibitor cocktail 2 (Sigma, P-5726). 40 μg of cell lysates were boiled in the presence of SDS sample buffer (0.5 M Tris-HCl, pH 6.8, 10% (w/v) SDS, 10% glycerol, 0.05% (w/v) bromophenol blue) for 5 minutes and loaded for electrophoresis on 10% SDS-PAGE. Proteins were transferred to nitrocellulose membranes (Cat# 162-0147) from Bio-Rad (Hercules, Calif.) using a semi-dry blotter (Bio-Rad). Membranes were blocked in blocking solution (5% non-fat dry milk in TBS containing 0.1% Tween 20) for 1 hour to prevent nonspecific binding and then washed three times with TBS-T (TBS containing 0.1% Tween 20). Membranes were incubated primary antibodies in TBS-T containing 5% bovine albumin (Sigma, Cat# A-7030) overnight at 4° C. The next day, membranes were washed three times with TBS-T and incubated with secondary antibody in TBS-T containing 5% non-fat dry milk for 1 hour. Membranes were washed extensively and enhanced chemiluminescence (ECL) detection assay was performed using SuperSignal West Dura kit from Pierce (Rockford, Ill.) according to the manufacturer's instructions.

### Example 1

### Construction of a Chimeric Receptor Capable of Mediating Osteoclast Formation and Function

To delineate functional motifs in the RANK cytoplasmic domain mediating osteoclast differentiation and function, a chimeric receptor comprising mouse TNFR1 external domain (residues 1-210 of SEQ ID NO:1) linked to the transmembrane and intracellular domains of mouse RANK (residues 210-625 of SEQ ID NO:2) was developed and tested as described in Liu et al., (2004) J. Biol. Chem., 279(52):54759-54769. BMMs from TNFR1 and R2 double knockout mice (TNFR1^{-/-}R2^{-/-}) were used to eliminate any possibility of signaling through TNF receptors. BMMs derived from TNFR1^{-/-}R2^{-/-} mice were infected with the retrovirus encoding the chimeric receptor and the cells expressing the chimera were selected with 1 μg/ml puromycin for 2 or 4 days. Flow cytometric analysis with antibody against the external domain of TNFR1 demonstrated that the chimera was not only expressed on the cell surface, but also the surface expression levels of the chimera were increased with the selection time.

Infected BMMs expressing the chimera were treated with M-CSF (22 ng/ml), M-CSF (22 ng/ml) plus RANKL (100 ng/ml), or M-CSF (22 ng/ml) plus TNFα (10 ng/ml). While infected BMMs treated with M-CSF alone remained in macrophage lineage, those treated with M-CSF and RANKL formed osteoclasts, indicating that the endogenous RANK in the infected cells is functional. When infected BMMs were treated with M-CSF and TNFα, they also formed osteoclasts, confirming that the chimeric receptor is working. As a negative control, uninfected BMMs failed to form osteoclasts in response to M-CSF and TNFα treatment. Notably, the infected cell culture treated with M-CSF alone also contained few TRAP-positive cells. This might be a result of over-expression of the chimera in a few BMMs, since over-expression of TNFR family members can lead to the self-activation of their signaling pathways. Functionally, these TRAP-positive cells may not be regarded as osteoclasts because they were still mononuclear. In addition, these TRAP-positive mononuclear cells failed to form any pits in the resorption assays. Thus, the formation of few TRAP-positive mononuclear cells in this culture does not undermine the chimera's potential as a tool to study RANK signaling. TNFα was typically used at a concentration of 10 ng/ml, however, lower TNFα concentrations (as low as 1 ng/ml) were still able to mediate osteoclast formation.

### Example 2

Identification of a 40-a.a. RANK Intracellular Segment Essential for Osteoclast Formation

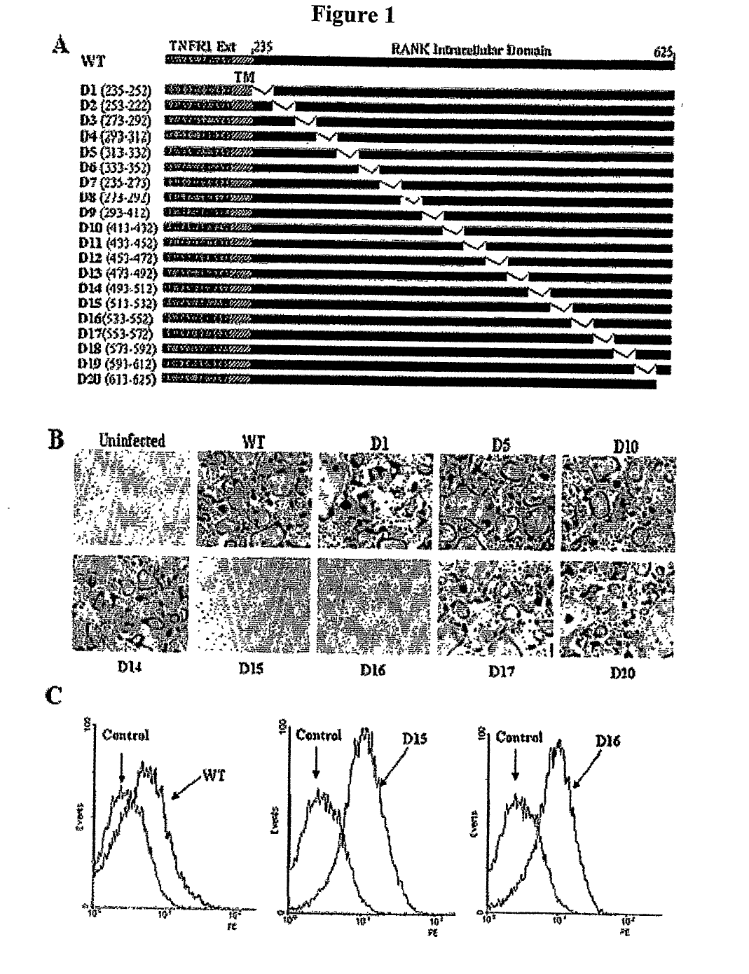

Mouse RANK has a 391-a.a. cytoplasmic domain. In order to identify novel RANK motifs involved in osteoclast formation, 20 internal deletion mutants were generated, named D1-D20 (FIG. 1A). In the D1 mutant, an 18 amino acid segment (residues 235-252 of SEQ ID NO:2) was deleted. In D2-D19, a 20 amino acid segment was deleted. In D20, the last 13 amino acids of the RANKL cytoplasmic domain were removed (FIG. 1A). Mutants were constructed in this manner since it was considered that small internal deletions (20 amino acids or smaller) may have minimal effects on the three-dimensional structure of the RANK cytoplasmic domain, which is essential for its function.

To determine whether these internal deletion mutants are capable of mediating osteoclast formation, the mutants and wild-type (WT) chimeras were expressed in primary BMMs derived from TNFR1^{-/-}R2^{-/-} using the retroviral system previously described (Liu et al., (2004) J. Biol. Chem. 279, 54759-54769). Infected BMMs were selected with 1 μg/ml puromycin for 2 days. Selected BMMs expressing the mutants were treated with M-CSF (22 ng/ml) and TNFα (10 ng/ml). All the internal deletion mutants except for D15 and D16 were able to mediate osteoclast formation, indicating that the regions deleted in D15 and D16 are functionally important for osteoclast formation. D15 contains a deletion of amino acids 513-532 of SEQ ID NO:2, and D16 contains a deletion of amino acids 533 to 552 of SEQ ID NO:2. FIG. 1B shows the negative control (uninfected BMMs), positive control (BMMs expressing the WT chimera), D15, D16 and several representatives of ineffective mutants (D1, D5, D10, D14, D17 and D18).

To confirm that the failure to form osteoclasts was not due to lack of expression, flow cytometry with an antibody against the external domain of TNFR1 demonstrated that the D15 and D16 mutants were expressed on the cell surface (FIG. 1C).

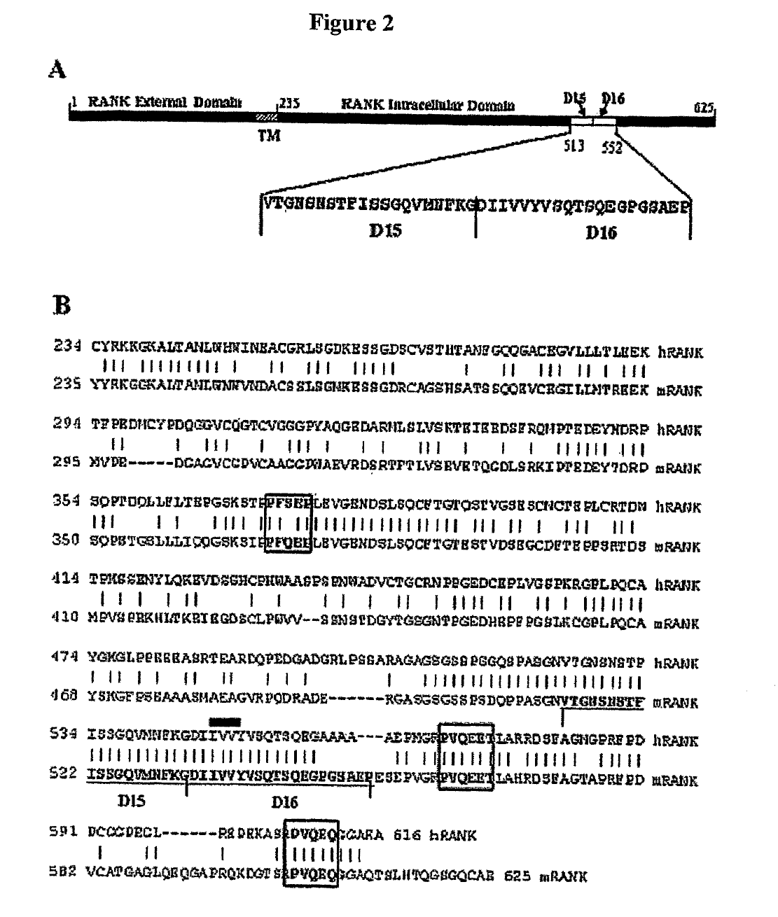

FIG. 2A shows the schematic location and amino acid sequence of the segments deleted in D15 and D16. Importantly, the segments deleted in D15 and D16 are located in a RANK cytoplasmic region that is highly conserved between human and mouse (FIG. 2B). Moreover, D15 and D16 do not overlap with the three functional RANK motifs identified previously (Liu et al., (2004) J. Biol. Chem. 279, 54759-54769; FIG. 2B).

### Example 3

### Identification of a 4-a.a Motif in the RANK Intracellular Region Essential for Osteoclast Formation

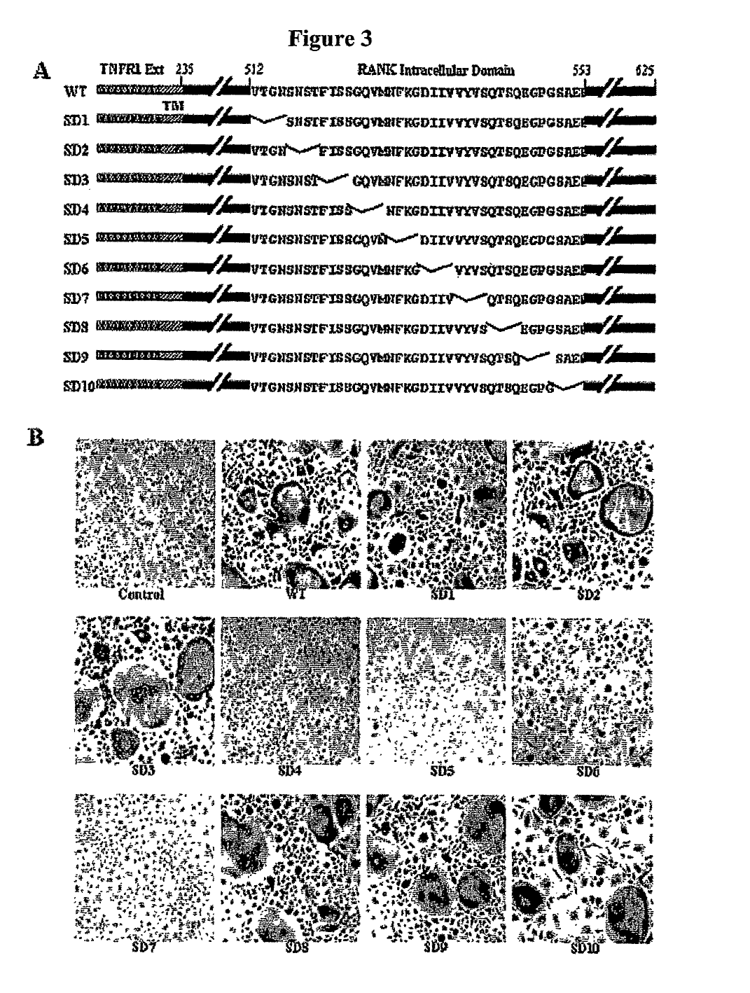

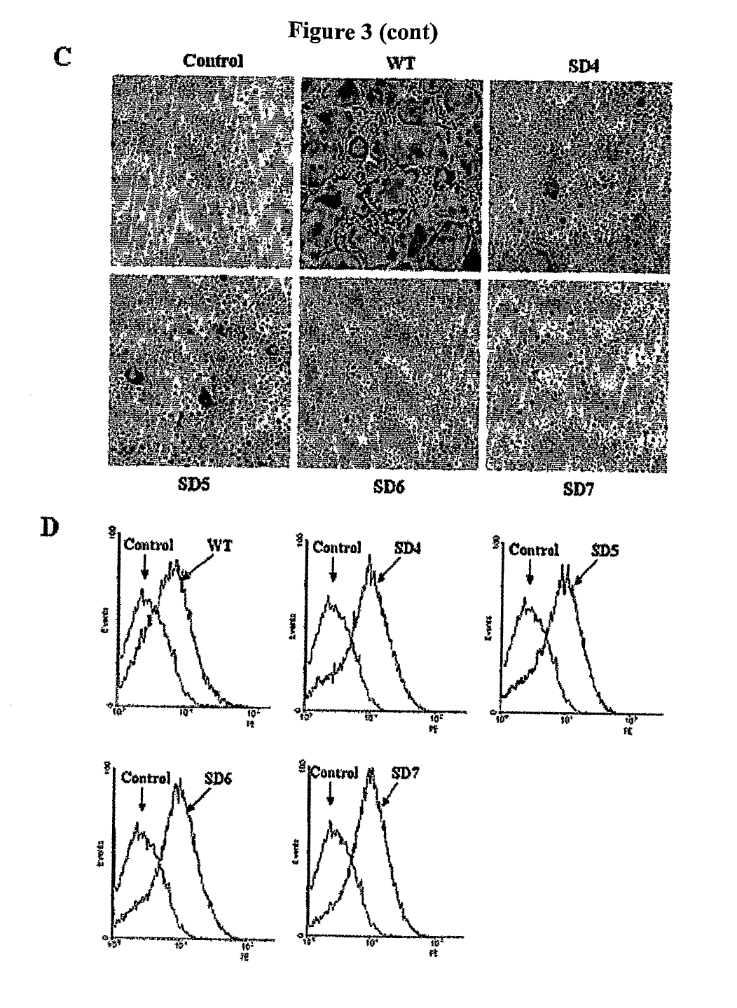

To further identify specific motifs in the 40-a.a. RANK segment that are involved in mediating osteoclast formation, ten more internal deletion mutants designated as SD1-SD10 were generated (FIG. 3A). In each of the 10 mutants, four amino acids in the 40-a.a. segment from amino acids 513 to 552 of SEQ ID NO:2 were deleted (FIG. 3A). Osteoclast formation assays were performed with these 10 mutants. As shown in FIG. 3B, when infected BMMs expressing these mutants were treated with M-CSF (22 ng/ml) and TNFα (10 ng/ml), SD4, SD5, SD6 and SD7 failed to mediate osteoclast formation, indicating that the residues deleted in these mutants are important for osteoclast formation.

The assays were repeated with higher TNFα concentrations (30 ng/ml). While SD6 and SD7 did not generate osteoclasts in response to the higher dose of TNFα, the SD4 and SD5 cultures contained a few osteoclasts. These results indicated that the residues deleted in SD6 and SD7 play more important roles in osteoclast formation than those residues deleted in SD4 and SD5. Flow cytometric analysis demonstrated that the SD4, SD5, SD6 and SD7 chimeras were expressed on the cell surface (FIG. 3D), confirming that the failure of these mutants to form osteoclasts is not due to a defect in their surface expression.

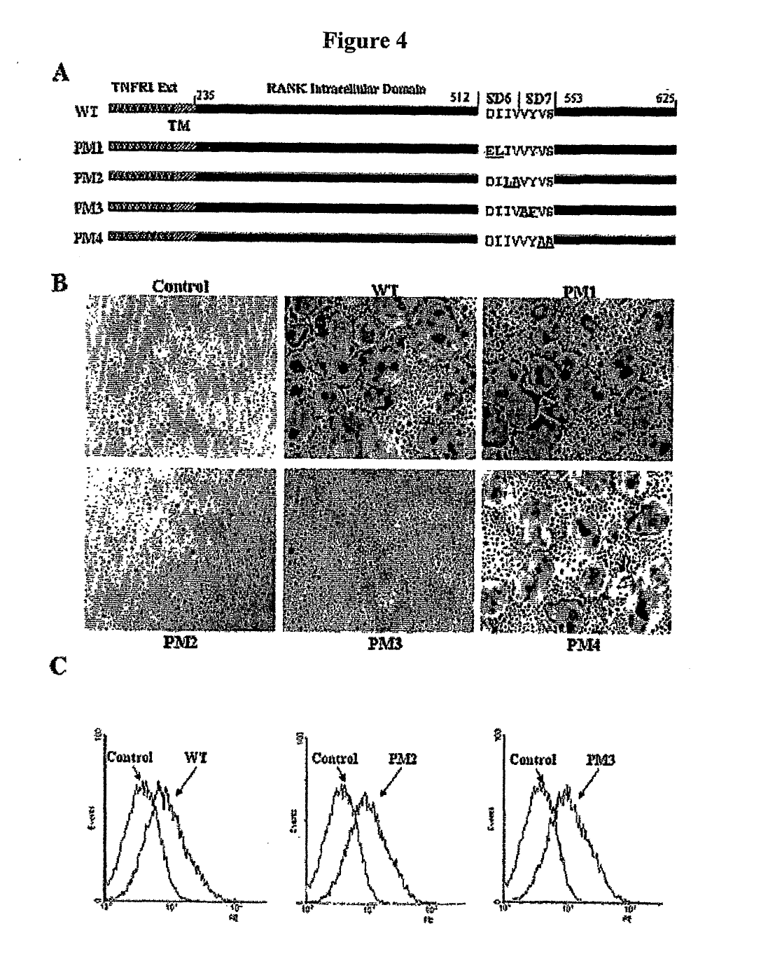

To identify specific amino acid residues in the 8-a.a. RANK segment encompassed by SD6 and SD7 (DIIVVYVS (residues 533-540 of SEQ ID NO:2)) that mediates osteoclast formation, four point mutation mutants were generated. FIG. 4A shows the schematic structure of the 4 mutant chimeric receptors constructed (PM1, PM2, PM3 and PM4). In each mutant, two residues were mutated. To minimize the effect of the point mutations on the three-dimensional structure of the RANK cytoplasmic domain, each of the 8 amino acid residues was mutated to an amino acid with similar chemical characteristics (e.g., similar chemical structure, polarity and charge). As shown in FIG. 4A, DIIVVYVS (residues 533-540 of SEQ ID NO:2) was mutated to ELIVVYVS (SEQ ID NO:12), DILAVYVS (SEQ ID NO:13), DIIVAFVS (SEQ ID NO:14), and DIIVVYAA (SEQ ID NO:15).

Osteoclast formation assays with these point mutants showed that only PM2 and PM3 failed to mediate osteoclast formation, revealing that four amino acid residues, IVVY⁵³⁵⁻⁵³⁸ (residues 535-538 of SEQ ID NO:2), are essential for osteoclast formation. Flow cytometric analysis confirmed that PM2 and PM3 were expressed on the cell surface (FIG. 4C).

### Example 4

### Activation of Known Signaling Pathways

It has been previously established that RANK mediates osteoclast formation and/or function by activating various intracellular signaling pathways including NF-KB (Hsu et al., (1999) Proc. Natl. Acad. Sci. U.S.A. 96, 3540-3545; Wong et al., (1999) Molecular Cell 4, 1041-1049), JNK (Hsu et al., (1999) Proc. Natl. Acad. Sci. U.S.A. 96, 3540-3545; Jimi et al., (1999) J. Immunol. 163, 434-442), ERK (Hsu et al., (1999) Proc. Natl. Acad. Sci. U.S.A. 96, 3540-3545; 30. Wei et al., (2002) J. Biol. Chem. 277, 6622-6630) and p38 (Wei et al. (2002) J. Biol. Chem. 277, 6622-6630; Matsumoto et al., (2000) J. Biol. Chem. 275, 31155-31161; Mansky et al., (2002) J. Biol. Chem. 277, 11077-11083), Akt (Wong et al., (1999) Molecular Cell 4, 1041-1049) and NFATc1 (Takayanagi et al., (2002) Developmental Cell 3, 889-901; Ishida et al., (2002) J. Biol. Chem. 277, 41147-41156). In addition, RANKL also stimulates the expression of c-fos (Takayanagi et al., (2002) Nature 416, 744-749), an essential factor for osteoclast formation (Blair et al., (1993) Clin. Orthop. Rel. Res. 294, 7-22). Upon the elucidation of this novel RANK motif, the possible role of this motif in activation of these signaling pathways was investigated.

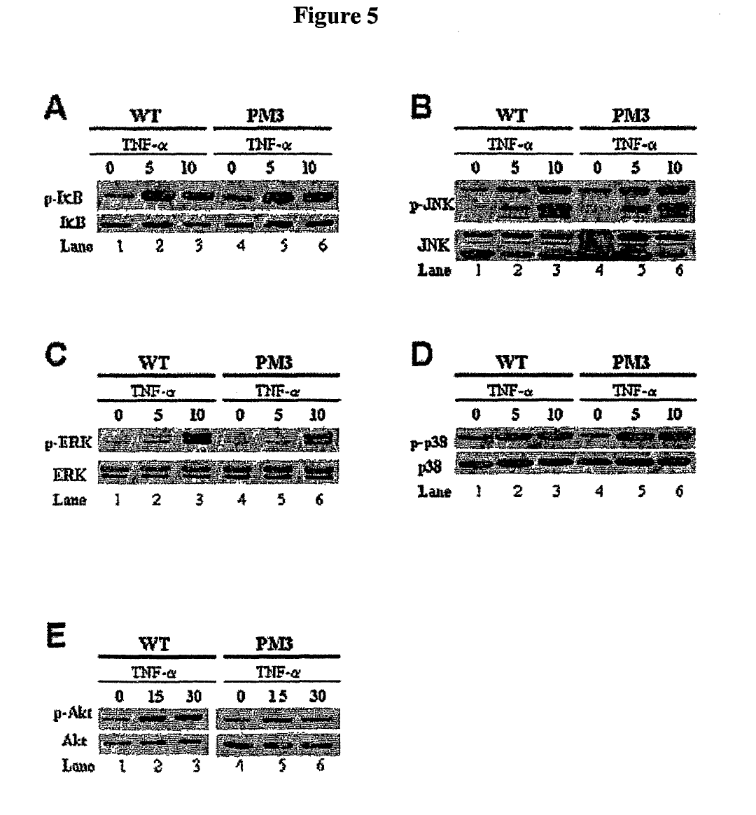

TNFR1^{-/-}R2^{-/-} BMMs were infected with virus encoding either the wild-type chimera or PM3. Infected cells were then treated with TNFα for 0, 5 and 10 minutes and the activation of the four pathways, NF-κB/IκB, JNK, ERK and p38, was determined by Western analysis (FIG. 5A-D). PM3 was able to activate NF-κB/IκB, JNK, ERK, and p38 (FIG. 5A-D). In addition, the infected cells were treated with TNFα for 0, 15 and 30 minutes and activation of Akt was determined, showing that PM3 was also able to activate Akt (FIG. 5E). Together, these data indicate that the novel motif in the RANK cytoplasmic domain regulates osteoclast formation by activating novel signaling pathways. Research is underway to identify the downstream signaling proteins that interact with the novel RANK motif, as well as to identify and develop therapeutics specific for this motif.

### Example 5

### Role of the Novel RANK Motif in Osteoclast Function

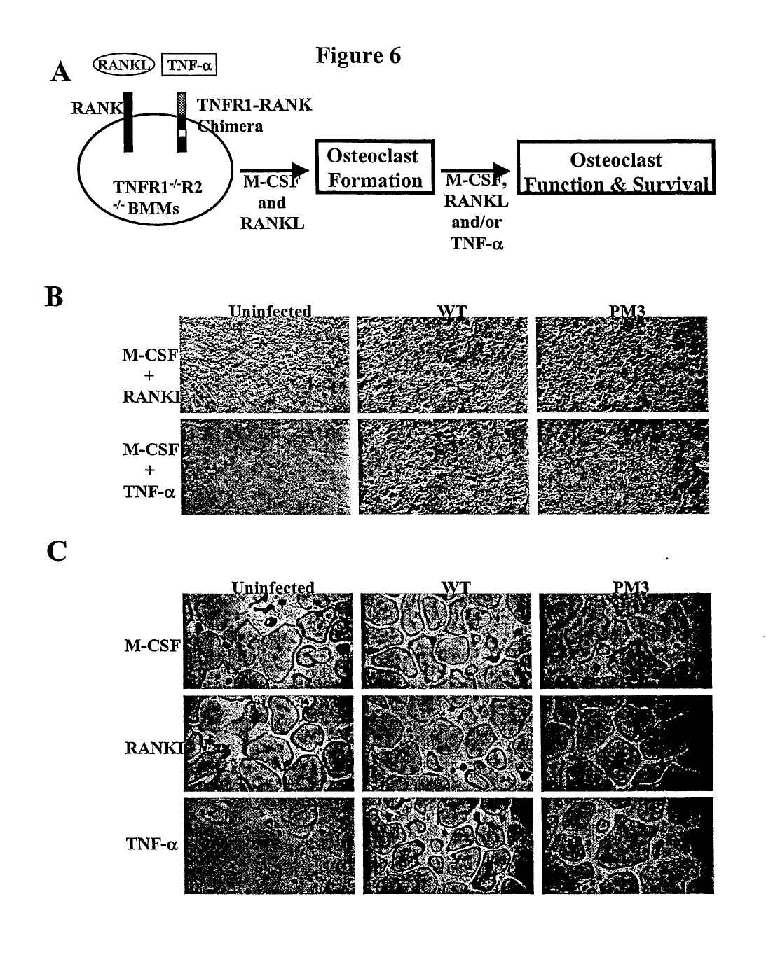

The role of the novel motif in osteoclast function, such as the ability to degrade bone, and survival was investigated. Since the mutation of the motif blocks osteoclast formation, the strategy shown in FIG. 6A was used to examine the role of the novel motif in osteoclast function and survival. TNFR1^{-/-}R2^{-/-} BMMs express both chimeric RNFR1-RANK and endogenous RANK. Dimerization of endogenous RANK in response to RANKL stimulation mediates osteoclast formation. Subsequently, the mature osteoclasts are treated with TNFα to examine the capacity of the chimeric receptor containing the mutated PM3 motif in modulating osteoclast function and survival.

Bone resorption assays are shown in FIG. 6B. Uninfected BMMs or BMMs infected with virus encoding PM3 were plated on bovine cortical bone slices and treated with M-CSF (44 ng/ml) and RANKL (100 ng/ml). After osteoclasts were formed, the cultures were then treated with M-CSF (44 ng/ml) plus TNFα (10 ng/ml) for approximately 3 days to determine whether the PM3 is able to mediate bone resorption. As positive control, separate cultures were treated with M-CSF and RANKL. The data showed that osteoclasts expressing either WT chimera or PM3 were very efficient in mediating bone resorption in response to TNFα stimulation, while those derived from uninfected BMMs generated significantly fewer bone resorption pits, indicating that the novel motif is not required for osteoclast function.

FIG. 6C shows osteoclast survival assays. Uninfected BMMs or BMMs infected with virus encoding PM3 were treated with M-CSF (44 ng/ml) and RANKL (100 ng/ml). After osteoclasts were formed, the cultures were then treated with M-CSF (44 ng/ml), RANKL (100 ng/ml) or TNFα (10 ng/ml) for 6 hours to determine whether PM3 is able to mediate osteoclast survival. The osteoclast survival assays demonstrated that osteoclasts expressing WT chimera or PM3 have a similar ability to promote osteoclast survival in response to TNFα stimulation, indicating that the novel motif is not required for osteoclast survival. As negative control, osteoclasts derived from uninfected BMMs failed to survive in response to TNFα stimulation.

These data suggest that the novel RANK motif plays an essential role in osteoclast formation but is not critical for osteoclast function and survival. Importantly, this data suggests that the motif is a useful target for an anti-resorptive therapeutic since it can be blocked to affect osteoclast formation without affecting function and survival of existing osteoclasts, and since the blockage of osteoclast formation makes osteoclast function and survival irrelevant issues.

The foregoing description of the invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

### Example 6

### Role of the Novel RANK Motif in Osteoclast Commitment

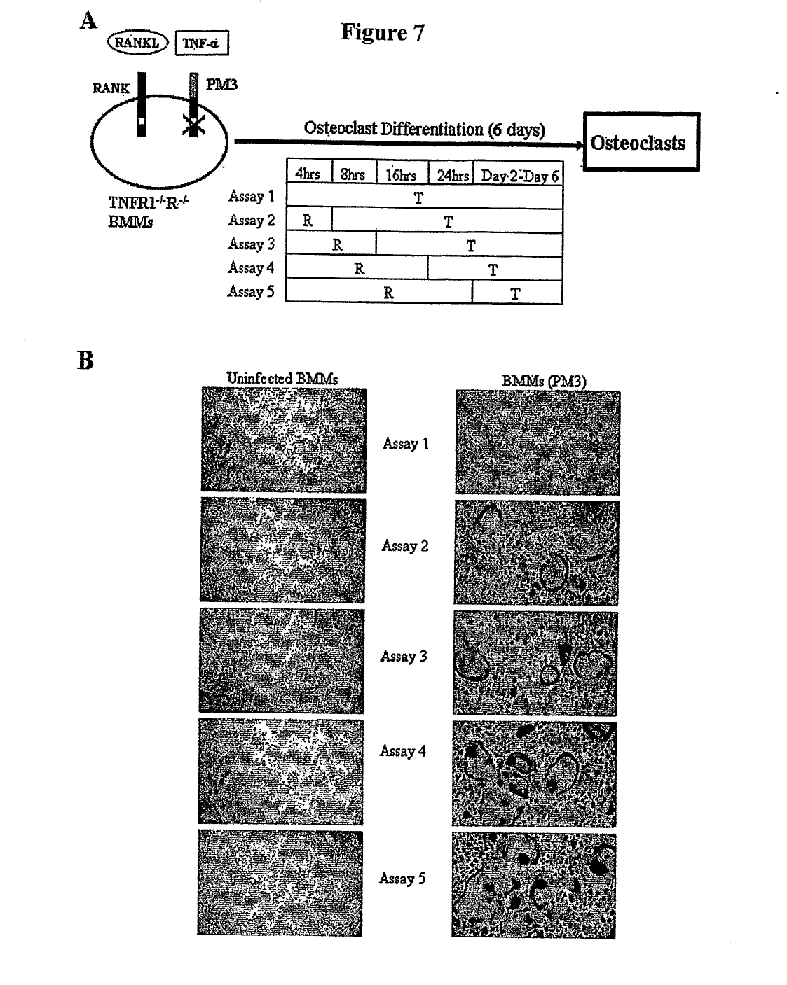

Experiments were performed as shown in FIG. 7 to further investigate the role of the novel motif in commitment of BMMs to the osteoclast lineage. As highlighted in FIG. 7A, BMMs infected with virus encoding PM3 express not only PM3 but also endogenous RANK. The novel motif is mutated in PM3 while it is functional in endogenous RANK.

The experiments were detailed in FIG. 7A. In assay 1, the infected or uninfected cells were treated with M-CSF (44 ng/ml) and TNF-α (10 ng/ml) throughout the 6 days of the osteoclastogenic process. In assay 2, the cells were treated with M-CSF (44 ng/ml) and RANKL (100 ng/ml) for 4 hours, and then the cultures were switched to M-CSF (44 ng/ml) and TNF-α (10 ng/ml) for the rest of the osteoclastogenic process. In assay 3, the cells were treated with M-CSF (44 ng/ml) and RANKL (100 ng/ml) for 8 hours, and then the cultures were switched to M-CSF (44/ml) and TNF-α (10 ng/ml) for the rest of the osteoclastogenic process. In assay 4, the cells were treated with M-CSF (44 ng/ml) and RANKL (100 ng/ml) for 16 hours, and then the cultures were switched to M-CSF (44 ng/ml) and TNF-α (10 ng/ml) for the rest of the osteoclastogenic process. In assay 5, the cells were treated with M-CSF (44 ng/ml) and RANKL (100 ng/ml) for 24 hours, and then the cultures were switched to M-CSF (44 ng/ml) and TNF-α (10 ng/ml) for the rest of the osteoclastogenic process.

The data as shown in FIG. 7B demonstrate that the treatment of BMMs with RANKL (targeting the endogenous RANK which is capable of activating the novel motif-mediated signaling) for only 4 hours can partially commit BMMs to osteoclast lineage (Assay 1, FIG. 7B). Moreover, 16 or 24-hour treatment of BMMs with RANKL can fully commit BMMs to osteoclast lineage (Assay 4 and 5, FIG. 7B). These data indicate that the novel motif-activated signaling pathway is required for committing BMMs to the osteoclast lineage.

### Example 7

### Identification of RANK-Mediated Signaling Pathways

An osteoclast precursor cDNA library suitable for two-hybrid screening was constructed using MATCHMAKER system from BD Biosciences Clontech (Palo Alto, Calif.). Specifically, full-length cDNAs prepared from mouse osteoclast precursors using CloneMiner™ cDNA Library Construction Kit (Invitrogen, Carlsbad, Calif.) were subcloned into the pGBKT7 AD Vector of MATCHMAKER Two-Hybrid System 3. A mouse cDNA region encoding a RANK fragment (residues 498-556 of SEQ ID NO: 2), which contains the IVVY (residues 535-538 of SEQ ID NO:2) motif and was used as bait in two-hybrid screening, was cloned into pGBKT7 DNA-BD Vector of MATCHMAKER Two-Hybrid System 3. Two rounds of two-hybrid screening (screening of 2×10⁶ clones) gave rise to approximately 30 positive clones/candidate genes.

8 clones with the highest affinity were sequenced. Of the 8 clones, there were four copies of BC080287; and one copy of each of NM_{—}008992, BX088552, BC082298, and BC003220. The sequences of the remaining clones are determined.

The involvement of these candidate genes in the novel motif-mediated osteoclast differentiation is determined using siRNA technology. Specifically, the siRNA target sequences for the candidate genes are selected using siRNA target finder available at Ambion, Inc (Austin, Tex.). In addition, interaction between the novel motif and the candidate genes is further assessed by techniques such as co-immunoprecipitation. Candidates that play a role in the osteoclasteogenic process and/or directly interact with the novel motif of the invention are useful for detecting modulation of at least one RANK-mediated signaling pathway.

Appendix

Amino acid sequence of mouse TNFR1 from NP_035739

(SEQ ID NO: 1)

| | |
|---|---|
| 1 | MGLPTVPGLL LSLVLLALLM GIHPSGVTGL VPSLGDREKR DSLCPQGKYV HSKNNSICCT |
| 61 | KCHKGTYLVS DCPSPGRDTV CRECEKGTFT ASQNYLRQCL SCKTCRKEMS QVEISFCQAD |
| 121 | KDTVCGCKEN QFQRYLSETH FQCVDCSPCF NGTVTIPCKE TQNTVCNCHA GFFLRESECV |
| 181 | PCSHCKKNEE CMKLCLPPPL ANVTNPQDSG TAVLLPLVIL LGLCLLSFIF ISLMCRYPRW |
| 241 | RPEVYSIICR DPVPVKEEKA GKPLTPAPSP AFSPTSGFNP TLGFSTPGFS SPVSSTPISP |
| 301 | IFGPSNWHFM PPVSEVVPTQ GADPLLYESL CSVPAPTSVQ KWEDSAHPQR PDNADLAILY |
| 361 | AVVDGVPPAR WKEFMRFMGL SEHEIERLEM QNGRCLREAQ YSMLEAWRRR TPRHEDTLEV |
| 421 | VGLVLSKMNL AGCLENILEA LRNPAPSSTT RLPR |

Amino acid sequence of mouse RANK from NP_033425

(SEQ ID NO:2)

| | |
|---|---|
| 1 | MAPRARRRRQ LPAPLLALCV LLVPLQVTLQ VTPPCTQERH YEHLGRCCSR CEPGKYLSSK |
| 61 | CTFTSDSVCL PCGPDEYLDT WNEEDKCLLH KVCDAGKALV AVDPGNHTAP RRCACTAGYH |
| 121 | WNSDCECCRR NTECAPGFGA QHPLQLNKDT VCTPCLLGFF SDVFSSTDKC KPWTNCTLLG |
| 181 | KLEAHQGTTE SDVVCSSSMT LRRPPKEAQA YLPSLIVLLL FISVVVVAAI IFGVYYRKGG |
| 241 | KALTANLWNW VNDACSSLSG NKESSGDRCA GSHSATSSQQ EVCEGILLMT REEKMVPEDG |
| 301 | AGVCGFVCAA GGPWAEVRDS RTFTLVSEVE TQGDLSRKIP TEDEYTDRPS QPSTGSLLLI |
| 361 | QQGSKSIPPF QEPLEVGEND SLSQCFTGTE STVDSEGCDF TEPPSRTDSM PVSFEKHLTK |
| 421 | EIEGDSCLPW VVSSNSTDGY TGSGNTPGED HEPFPGSLKC GPLPQCAYSM GFPSEAAASM |
| 481 | AEAGVRPQDR ADEKGASGSG SSPSDQPPAS GNVTGNSNST FISSGQVMNF KGDIIVVYVS |
| 541 | QTSQEGPGSA EPESEPVGRP VQEETLAHRD SFAGTAPRFP DVCATGAGLQ EQGAPRQKDG |
| 601 | TSRPVQEQGG AQTSLHTQGS GQCAE |

Amino acid sequence of human RANK from NP_003830

(SEQ ID NO:3)

| | |
|---|---|
| 1 | MAPRARRRRP LFALLLLCAL LARLQVALQI APPCTSEKHY EHLGRCCNKC EPGKYMSSKC |
| 61 | TTTSDSVCLP CGPDEYLDSW NEEDKCLLHK VCDTGKALVA VVAGNSTTPR RCACTAGYHW |
| 121 | SQDCECCRRN TECAPGLGAQ HPLQLNKDTV CKPCLAGYFS DAFSSTDKCR PWTNCTFLGK |
| 181 | RVEHHGTEKS DAVCSSSLPA RKPPNEPHVY LPGLIILLLF ASVALVAAII FGVCYRKKGK |
| 241 | ALTANLWHWI NEACGRLSGD KESSGDSCVS THTANFGQQG ACEGVLLLTL EEKTFPEDMC |
| 301 | YPDQGGVCQG TCVGGGPYAQ GEDARMLSLV SKTEIEEDSF RQMPTEDEYM DRPSQPTDQL |
| 361 | LFLTEPGSKS TPPFSEPLEV GENDSLSQCF TGTQSTVGSE SCNCTEPLCR TDWTPMSSEN |
| 421 | YLQKEVDSGH CPHWAASPSP NWADVCTGCR NPPGEDCEPL VGSPKRGPLP QCAYGMGLPP |
| 481 | EEEASRTEAR DQPEDGADGR LPSSARAGAG SGSSPGGQSP ASGNVTGNSN STFISSGQVM |
| 541 | NFKGDIIVVY VSQTSQEGAA AAAEPMGRPV QEETLARRDS FAGNGPRFPD PCGGPEGLRE |
| 601 | PEKASRPVQE QGGAKA |

What is claimed is:

1. A variant RANK polypeptide comprising at least one mutation at an amino acid residue corresponding to an amino acid residue selected from the group consisting of I535, V536, V537, Y538, and any combination of the foregoing, of SEQ ID NO:2.

2. The polypeptide of claim 1, wherein the variant RANK polypeptide has decreased activity of at least one RANK-mediated signaling pathway

3. The polypeptide of claim 1, wherein the mutation is selected from the group consisting of a deletion, substitution, addition, and any combination of the foregoing, of an amino acid residue.

4. A nucleic acid encoding a RANK polypeptide comprising at least one mutation at an amino acid residue corresponding to an amino acid residue selected from the group consisting of I535, V536, V537, Y538, and any combination of the foregoing, of SEQ ID NO:2.

5. A chimeric polypeptide comprising an non-RANK extracellular domain and at least 20 contiguous amino acids of a RANK intracellular domain, comprising residues corresponding to amino acids 535-538 of SEQ ID NO:2.

6. The polypeptide of claim 5, wherein the RANK intracellular domain comprises at least one mutation at an amino acid residue corresponding to an amino acid residue selected from the group consisting of I535, V536, V537, Y538, and any combination of the foregoing, of SEQ ID NO:2.

7. A method for identifying a compound capable of modulating osteoclast cell differentiation, comprising:
a) providing an osteoclast precursor cell comprising a receptor comprising a RANK polypeptide,
b) contacting the osteoclast precursor cell with a test compound and a ligand for the receptor, wherein the test compound interacts with one or more amino acids corresponding to an amino acid residue of 535-538 of SEQ ID NO:2, and
c) determining whether osteoclast formation has been modulated, said modulation being an indication that the compound modulates osteoclast cell differentiation.

8. The method of claim 7, wherein the RANK polypeptide is a mouse polypeptide.

9. The method of claim 7, wherein the RANK polypeptide is a human polypeptide.

10. The method of claim 7, wherein the receptor is a chimeric polypeptide.

11. The method of claim 10, wherein the chimeric polypeptide comprises a RANK intracellular domain.

12. The method of claim 11, wherein the RANK intracellular domain comprises at least contiguous 20 amino acids of a RANK polypeptide, wherein the RANK polypeptide comprises residues corresponding to amino acids 535-538 of SEQ ID NO:2.

13. The method of claim 10, wherein the chimeric polypeptide comprises a non-RANK polypeptide comprising an extracellular domain.

14. The method of claim 13, wherein the non-RANK polypeptide is a TNF receptor.

15. The method of claim 14, wherein the ligand is TNFα.

16. The method of claim 7, wherein the rate of osteoclast formation is decreased.

17. A method for identifying a compound capable of modulating RANK activity, said method comprising:
a) inducing oligomerization of a receptor comprising a RANK polypeptide in the presence or absence of a test compound, wherein the test compound interacts with one or more amino acids corresponding to an amino acid residue of 535-538 of SEQ ID NO:2, and
b) detecting modulation of at least one RANK-mediated signaling pathway in said cell after said oligomerization, said activation level being an indication that the compound modulates activity of RANK.

18. The method of claim 17, wherein the RANK polypeptide is a mouse polypeptide.

19. The method of claim 17, wherein the RANK polypeptide is a human polypeptide.

20. The method of claim 17, wherein the receptor is a chimeric polypeptide.

21. The method of claim 20, wherein the chimeric polypeptide comprises at least contiguous 20 amino acids of a RANK polypeptide, wherein the RANK polypeptide comprises residues corresponding to amino acids 535-538 of SEQ ID NO:2.

22. The method of claim 17, wherein oligomerization occurs in an osteoclast precursor cell, and the activation level is determined by detecting osteoclast formation.

23. The method of claim 22, wherein osteoclast formation is decreased.

24. The method of claim 17, wherein the compound down-regulates at least one RANK-mediated signaling pathway.

25. A method for identifying a compound capable of modulating RANK activity, comprising:
a) providing a cell comprising a receptor comprising a RANK polypeptide,
b) contacting the cell with a test compound and a ligand for the receptor, wherein the test compound interacts with one or more amino acids corresponding to an amino acid residue of 535-538 of SEQ ID NO:2, and
c) determining whether a RANK-mediated signaling pathway has been modulated, said modulation being an indication that the compound modulates RANK activity.

26. The method of claim 25, wherein the RANK polypeptide is a mouse polypeptide.

27. The method of claim 25, wherein the RANK polypeptide is a human polypeptide.

28. The method of claim 25, wherein the RANK polypeptide is a chimeric polypeptide.

29. The method of claim 28, wherein the chimeric polypeptide comprises a RANK intracellular domain.

30. The method of claim 29, wherein the RANK intracellular domain comprises at least contiguous 20 amino acids of a RANK polypeptide, wherein the RANK polypeptide comprises residues corresponding to amino acids 535-538 of SEQ ID NO:2.

31. The method of claim 28, wherein the chimeric polypeptide comprises a non-RANK polypeptide comprising an extracellular domain.

32. The method of claim 31, wherein the non-RANK polypeptide is a TNF receptor.

33. The method of claim 32, wherein the ligand is TNFα.

34. The method of claim 25, wherein the cell is an osteoclast precursor cell, and wherein contact with the compound reduces osteoclast formation.

35. A method for identifying a compound capable of inhibiting RANK activity, said method comprising:
a) inducing oligomerization of a chimeric transmembrane protein in an osteoclast precursor cell in the presence or absence of a test compound, said chimeric protein comprising a non-RANK extracellular domain and a RANK intracellular domain, wherein the test compound interacts with one or more amino acids corresponding to an amino acid residue of 535-538 of SEQ ID NO:2; and
b) detecting activation level of at least one RANK-mediated signaling pathway in said cell after said oligomerization, wherein a reduction in the activity level in the presence of said molecule compared to that in the absence of said molecule is indicative of the ability of said molecule to inhibit RANK activity.

36. A process for making a compound that decreases osteoclast cell differentiation, comprising:
a) carrying out the method of any of claims 7, 17, 25, or 35 to identify a compound that decreases osteoclast cell differentiation, and
b) manufacturing the compound.

37. A method of improving bone mass in an individual in need thereof, comprising administering to the individual a therapeutically effective amount of a compound that decreases differentiation of a osteoclast precursor cell to an osteoclast, wherein the compound interacts with one or more amino acids corresponding to residues 535-538 of SEQ ID NO:2.

38. A method of improving bone mass in an individual in need thereof, comprising administering to the individual a therapeutically effective amount of a compound that inhibits the activity of RANK in an osteoclast cell, wherein the compound interacts with one or more amino acids corresponding to residues 535-538 of SEQ ID NO:2.

39. The method of claim 37 or claim 38, wherein the individual has a bone-related disorder selected from the group consisting of osteoporosis, rheumatoid arthritis, cancer-induced bone lesions, T-cell or B-cell malignancies, or other cancers or bone disorders.


SEQUENCE LISTING (25 sequences)

SEQ ID NO: 1 - protein, 454 aa, Mus musculus
MGLPTVPGLLLSLVLLALLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTAVLLPLVILLGLCLLSFIFISLMCRYPRWRPEVYSIICRDPVPVKEEKAGKPLTPAPSPAFSPTSGFNPTLGFSTPGFSSPVSSTPISPIFGPSNWHFMPPVSEVVPTQGADPLLYESLCSVPAPTSVQKWEDSAHPQRPDNADLAILYAVVDGVPPARWKEFMRFMGLSEHEIERLEMQNGRCLREAQYSMLEAWRRRTPRHEDTLEVVGLVLSKMNLAGCLENILEALRNPAPSSTTRLPR

SEQ ID NO: 2 - protein, 625 aa, Mus musculus
MAPRARRRRQLPAPLLALCVLLVPLQVTLQVTPPCTQERHYEHLGRCCSRCEPGKYLSSKCTPTSDSVCLPCGPDEYLDTWNEEDKCLLHKVCDAGKALVAVDPGNHTAPRRCACTAGYHWNSDCECCRRNTECAPGFGAQHPLQLNKDTVCTPCLLGFFSDVFSSTDKCKPWTNCTLLGKLEAHQGTTESDVVCSSSMTLRRPPKEAQAYLPSLIVLLLFISVVVVAAIIFGVYYRKGGKALTANLWNWVNDACSSLSGNKESSGDRCAGSHSATSSQQEVCEGILLMTREEKMVPEDGAGVCGPVCAAGGPWAEVRDSRTFTLVSEVETQGDLSRKIPTEDEYTDRPSQPSTGSLLLIQQGSKSIPPFQEPLEVGENDSLSQCFTGTESTVDSEGCDFTEPPSRTDSMPVSPEKHLTKEIEGDSCLPWVVSSNSTDGYTGSGNTPGEDHEPFPGSLKCGPLPQCAYSMGFPSEAAASMAEAGVRPQDRADEKGASGSGSSPSDQPPASGNVTGNSNSTFISSGQVMNFKGDIIVVYVSQTSQEGPGSAEPESEPVGRPVQEETLAHRDSFAGTAPRFPDVCATGAGLQEQGAPRQKDGTSRPVQEQGGAQTSLHTQGSGQCAE

SEQ ID NO: 3 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 4 - DNA, 30 bp, Artificial Sequence
  Description of Artificial Sequence Synthetic primer
aacttcaagggtggactcatcgtggtgtat

SEQ ID NO: 5 - DNA, 30 bp, Artificial Sequence
  Description of Artificial Sequence Synthetic primer
ttgaagttcccacttgagtagcaccacata

SEQ ID NO: 6 - DNA, 30 bp, Artificial Sequence
  Description of Artificial Sequence Synthetic primer
aagggtgacatcctcgcggtgtatgtcagc

SEQ ID NO: 7 - DNA, 30 bp, Artificial Sequence
  Description of Artificial Sequence Synthetic primer
ttcccactgtaggagcgccacatacagtcg

SEQ ID NO: 8 - DNA, 30 bp, Artificial Sequence
  Description of Artificial Sequence Synthetic primer
gacatcatcgtggcgtttgtcagccagacc

SEQ ID NO: 9 - DNA, 30 bp, Artificial Sequence
  Description of Artificial Sequence Synthetic primer
ctgtagtagcaccgcaaacagtcggtctgg

SEQ ID NO: 10 - DNA, 30 bp, Artificial Sequence
  Description of Artificial Sequence Synthetic primer
atcgtggtgtatgccgcccagacctcgcag

SEQ ID NO: 11 - DNA, 30 bp, Artificial Sequence
  Description of Artificial Sequence Synthetic primer
tagcaccacatacggcgggtctggagcgtc

SEQ ID NO: 12 - protein, 8 aa, Mus musculus
ELIVVYVS

SEQ ID NO: 13 - protein, 8 aa, Mus musculus
DILAVYVS

SEQ ID NO: 14 - protein, 8 aa, Mus musculus
DIIVAFVS

SEQ ID NO: 15 - protein, 8 aa, Mus musculus
DIIVVYAA

SEQ ID NO: 16 - protein, 36 aa, Mus musculus
SNSTFISSGQVMNFKGDIIVVYVSQTSQEGPGSAEP

SEQ ID NO: 17 - protein, 36 aa, Mus musculus
VTGNFISSGQVMNFKGDIIVVYVSQTSQEGPGSAEP

SEQ ID NO: 18 - protein, 36 aa, Mus musculus
VTGNSNSTGQVMNFKGDIIVVYVSQTSQEGPGSAEP

SEQ ID NO: 19 - protein, 36 aa, Mus musculus
VTGNSNSTFISSNFKGDIIVVYVSQTSQEGPGSAEP

SEQ ID NO: 20 - protein, 36 aa, Mus musculus
VTGNSNSTFISSGQVMDIIVVYVSQTSQEGPGSAEP

SEQ ID NO: 21 - protein, 36 aa, Mus musculus
VTGNSNSTFISSGQVMNFKGVYVSQTSQEGPGSAEP

SEQ ID NO: 22 - protein, 36 aa, Mus musculus
VTGNSNSTFISSGQVMNFKGDIIVQTSQEGPGSAEP

SEQ ID NO: 23 - protein, 36 aa, Mus musculus
VTGNSNSTFISSGQVMNFKGDIIVVYVSEGPGSAEP

SEQ ID NO: 24 - protein, 36 aa, Mus musculus
VTGNSNSTFISSGQVMNFKGDIIVVYVSQTSQSAEP

SEQ ID NO: 25 - protein, 36 aa, Mus musculus
VTGNSNSTFISSGQVMNFKGDIIVVYVSQTSQEGPG